(19) United States
(12) Patent Application Publication
(10) Pub. No.: US 2011/0263843 A1
(43) Pub. Date: Oct. 27, 2011

(54) MICROWAVE RADIATING DEVICE, CONNECTING TYPE MICROWAVE RADIATING DEVICE, AND METHODS OF PRODUCING SUGAR INGREDIENT FROM PLANT MATERIALS

(75) Inventors: Takashi Watanabe, Kyoto (JP); Naoki Shinohara, Kyoto (JP); Tomohiko Mitani, Kyoto (JP); Masafumi Oyadomari, Kyoto (JP); Yasunori Ohashi, Kyoto (JP); Pradeep Verma, Kyoto (JP); Takahiko Tsumiya, Osaka (JP); Hisayuki Sego, Osaka (JP); Yasuhiro Takami, Osaka (JP)

(73) Assignees: Japan Chemical Engineering & Machinery Co., Ltd., Osaka-shi (JP); Kyoto University, Kyoto-shi (JP)

(21) Appl. No.: 12/737,596

(22) PCT Filed: Jul. 28, 2009
(86) PCT No.: PCT/JP2009/063398

§ 371 Date: May 24, 2011

(30) Foreign Application Priority Data
Jul. 28, 2008 (JP) 2008-193434
Jan. 23, 2009 (JP) 2009-013689
Mar. 24, 2009 (JP) 2009-072933

(51) Int. Cl. C07H 1/08; B01J 19/12

(52) U.S. Cl. 536/128; 422/186.29

(57) ABSTRACT

The present invention provides a microwave radiating device which treats a radiated material by microwave irradiation; and further provides a method of producing a sugar ingredient from a plant material.

The microwave radiating device of the present invention is equipped with an irradiating means which has a microwave radiating source, and containers which accept radiated material inside. Further, the container of the device has supply means which provides radiated material to the container, and discharge means which discharges radiated material from the container, and a microwave receiving section which makes microwaves that irradiate from radiating means, penetrate into the container through the medium of dielectric material. Further, there is provided at least one layer that is composed of non-dielectric material, between the atmosphere of the inner side and the outer side of the container. Yet, irradiating means and the above container are connected in a removable mode.

This application claims priority under 35 U.S.C. §120 and §365(c) as a continuation of international application number PCT/JP2009/063398, filed Jul. 28, 2009, entitled “MICROWAVE IRRATIATION DEVICE, LINKED MICROWAVE IRRADIATION DEVICE, AND METHOD OF MANUFACTURING GLYCOCOMPONENT FROM PLANT MATERIAL,” which claims priority to: Japanese Patent Application No. JP2008-193434 filed on Jul. 28, 2008; JP2009-013689 filed on Jan. 23, 2009; and JP2009-072933 filed on Mar. 24, 2009, which are incorporated herein by reference.

### FIELD OF THE INVENTION

The present invention is related to a microwave radiating device which may treat a large amount of radiated material by microwave irradiation. The invention is also related to a connecting type microwave radiating device which is a combination of microwave radiating devices. Further, the present invention is related to a method which is able to produce a sugar ingredient efficiently as well as preventing over-decomposition of saccharides at a condition of low environmental impact, and is able to get polysaccharides by sufficient saccharification even at a small amount of enzyme.

### BACKGROUND

When microwave irradiates certain materials, the movement of the molecules of the material is activated. These various effects of microwave radiating treatment are well known. Accordingly, microwave treatment provides extensive uses such as heating, sterilization, dehydration, extraction, or organic synthesis of materials in the field of organic chemistry or inorganic chemistry, biochemistry etc.

A traditional microwave radiating device which is used presently is shown in FIG. 5. To give shape to this traditional device, the radiated material is first carried to a reacting tube 404 which is made of dielectric material such as ceramic, by a pump 402. Microwave is generated by microwave radiating source 406, and passes through the inner side of a waveguide 408 and reaches a heating device 410 which is set around the reacting tube 404. The microwave penetrates the dielectric material, and the radiated material at the inner side of the reacting tube 404 is subjected to microwave irradiation. Moreover, because the microwave is reflected diffusely, the radiated material is irradiated homogeneously.

Further, there are also other microwave radiating devices with different constructions which may react at high temperature and high pressure.

### SUMMARY OF THE INVENTION

The structure of a traditional microwave radiating device is complicated, so the periodical maintenance becomes a great burden. Especially, to exchange the dielectric material used for penetration of the microwave takes much time and costs a lot. Furthermore, the microwave radiating device is out of use during the maintenance time. The fact is that if the tube used in the microwave radiating device is made of dielectric material, the above problem can not be solved because there is an upper limit set on reacting temperature and pressure. Therefore it is desired to provide a microwave radiating device which is able to perform the reaction under severe conditions such as high temperature or pressure, decompression, vacuum etc., and be able to solve the maintenance problem mentioned above.

The microwave radiating devices in the prior art have dielectric material used in the microwave penetrating portion. If the above portion of the microwave radiating device is out of order, repair would be very difficult because the device has to be dismantled. Moreover, the reacting container and the waveguide are manufactured as a unit, it is not easy to change the penetration of microwave, e.g. it may not correspond to a user's desire when a user hopes to change the penetration of microwave to optimize the reaction condition.

Further, when a user wants to increase the amount of microwave treatment, the only way is to buy a new microwave radiating device. It is not economical.

In light of the above problems, the objects of the present invention are to provide a microwave radiating device, which realizes microwave treatment at severe conditions of high temperature or pressure, decompression, vacuum etc., and to provide a microwave radiating device flexibly and rapidly corresponding to a user's various needs such as short maintenance time, optimization of microwave penetration, addition of more microwave radiating devices etc.

Furthermore, the present invention provides a method of producing sugar ingredients from plant materials, wherein the method is able to produce sugar ingredients efficiently as well as preventing over-decomposition of saccharides at a condition of low environmental impact, and is able to obtain polysaccharides by sufficient saccharification even at a small amount of enzyme; and the present invention provides a method of producing sugar ingredients from plant materials, wherein the method is able to produce sugar ingredients efficiently as well as preventing over-decomposition of saccharides at a condition of low environmental impact, and is able to use water as a dispersion medium, and is able to obtain polysaccharides by sufficient saccharification even at a small amount of enzyme.

The microwave radiating device of the present invention is equipped with irradiating means which has a microwave radiating source, and containers which accept radiated material inside. The container has supply means which provides radiated material to the container, and discharge means which discharges radiated material from the container, and a microwave receiving section which makes microwave irradiated from radiating means that penetrate into the container through the medium of dielectric material. There is provided at least one layer composed of non-dielectric material between the atmosphere of the inner side and the outer side of the container. Yet, irradiating means and container are connected in a removable mode.

Further, the microwave radiating device of the present invention is favorable in that dielectric material and sheet shaped parts are laminated at the opening direction of the microwave receiving section of the container.

Further, the microwave radiating device of the present invention is favorable in that the above dielectric material is sandwiched by at least 2 pieces of sheet shaped parts.

Further, the microwave radiating device of the present invention may comprise dielectric materials which are selected from the groups consisting of quartz glass, fluorocarbon polymers, ceramics, alumina, sapphire, and diamond; and/or non-dielectric materials which are selected from the groups consisting of stainless steel, aluminium, titanium, nickel, gold, and silver.

Further, the connecting type microwave radiating device of the present invention may be a combination device that combines more than two of the microwave radiating devices of the present invention. A removable structure between a discharge means of one microwave radiating device and a supply means of a neighboring microwave radiating device are able to attach to or remove from each other. When the discharge means connects to the supply means, various containers of microwave radiating devices configure as a unit and form a flow way of radiated material.

Furthermore, according to the present invention, the method of producing sugar ingredients from plant material is able to produce sugar ingredient efficiently as well as preventing over-decomposition of saccharides by comprising a separation process wherein a mixture of decomposed products of lignin and a sugar ingredient which at least contains solid-state polysaccharides is obtained by means of microwave irradiation under the existence of a Lewis acid catalyst to a plant material which at least has lignocellulose. Moreover, the obtained sugar ingredient here may be saccharized sufficiently even at a small amount of enzyme.

Furthermore, according to another method of the present invention, in the separation process a 2000 MHz-6000 MHz microwave is used. Further according to another method, microwave irradiates 1 minute-60minutes. Further according to another method, the plant material is heated to 80° C.-240° C. when microwave irradiates. According to these conditions it is able to produce a sugar ingredient more efficiently.

Furthermore, in another method of the present invention, in the separation process the Lewis acid catalyst may be selected from the groups consisting of aluminium chloride, aluminium bromide, aluminium sulfate, iron(III) chloride, zinc chloride, zinc bromide, and zinc sulfate.

Furthermore, according to the present invention, the method of producing a sugar ingredient from a plant material, is able to produce the sugar ingredient efficiently as well as inhibiting over-decomposition of saccharides by comprising a separation process wherein a mixture of decomposed products of lignin and a sugar ingredient which at least contains solid-state polysaccharides by means of microwave irradiation of a plant material which has lignocellulose with molybdic acid ion, ammonium ion, and peroxides. Moreover, the obtained sugar ingredient here may be saccharized sufficiently even at a small amount of enzyme.

Furthermore, in another method of the present invention, in the separation process a 2000 MHz-6000 MHz microwave is used. Further in another method, microwave irradiates 1 minute-60 minutes. Further in another method, the plant material is heated to 50° C.-240° C. when the microwave irradiates. According to these conditions it is able to produce a sugar ingredient more efficiently.

Furthermore, in the separation process of another method of the present invention, microwave irradiates plant material in water. Thus the time, trouble, or cost of posterior treatment would be decreased, also the environmental impact would be reduced.

The microwave radiating device of the present invention has at least one layer made of non-dielectric material, located between the inner side and outer side of the container where the reaction occurs; the layer may prevent microwave from escaping to outer atmosphere. Further, there is provided a broader usage than traditional microwave radiating devices because the microwave radiating device of the present invention is able to react at severe conditions such as high temperature or pressure, decompression, vacuum etc. Especially when carrying radiated material under pressure the microwave radiating device of the present invention will endure the high pressure which occurs inside of the reacting tube of the device. Further, the maintenance of the microwave receiving section which has a dielectric material is able to be accomplished easily and rapidly by removing the irradiating means from the container.

The receiving section of the microwave radiating device of the present invention is comprised of a lamination of dielectric material and sheet shaped parts, and manufacturing is easier compared to an integrated microwave receiving section. Further, modification of the penetration amount on the radiated material is easy by adjusting the penetration of the sheet shaped parts. Further, if sheet shaped parts with various properties are prepared previously it is able to optimize the reaction condition by only exchanging the sheet shaped parts. The exchange of the sheet shaped parts is able to be accomplished easily and rapidly by removing the irradiating means from the container.

The microwave radiating device of the present invention has at least more than two pieces of sheet shaped parts so it is possible to adjust penetration of the microwave precisely. Further, during the time of removing the irradiating means and the container, the above dielectric materials are hardly damaged because the dielectric materials are sandwiched by sheet shaped parts.

The connecting type microwave radiating device of the present invention is able to provide devices to meet various user's needs. Especially when a user who is using the microwave radiating device of the present invention wants to increase the treatment amount of the microwave, he just purchases necessary microwave radiating devices and connects them without changing the whole device. Thus it is extremely economic. Further, each device is connected in a removable state; at the time of maintenance just take the old device away and replace a new one, then connect again; the whole device is able to be used continuously.

The method of producing a sugar ingredient from a plant material according to the present invention is able to separate the sugar ingredient at a lower reaction temperature than that of a traditional method, thus may efficiently produce the sugar ingredient which contains solid-state polysaccharides as well as inhibit over-decomposition of saccharides. Such inhibition of over-decomposition will reduce formation of furfural which impedes fermentation. Further, producing of the sugar ingredient according to the present invention is able to decrease cost and reduce environmental impact because the reaction will proceed at a lower temperature than that of the traditional method and even the enzyme is in a small quantity. Particularly, compared to the traditional method the present invention is able to conduct efficiently the production of a sugar ingredient from a plant material of a conifer source.

The decomposed products of lignin obtained from the process of producing a sugar ingredient according to the present invention may be used in the production of useful chemical substances such as aromatic compounds etc. The amounts and types of the decomposed products of lignin according to the present invention are more than those of the traditional methods, and are suitable for production of chemical substances.

The produced sugar ingredient according to the present invention may contain low molecule saccharides such as monosaccharide or oligosaccharide etc. in addition to solid-state polysaccharides. Such low molecule saccharides are able to be applied to the production of useful chemical substances. As described above, various chemical substances are able to be produced from a plant material according to the present invention; this realization of biorefinery will be a great contribution of the present invention.

Further, among the separated sugar ingredients according to the present invention, low molecule saccharides are obtained from solid-state polysaccharides by conducting a saccharification treatment. Particularly, compared with the traditional method, the enzymatic saccharification treatment using cellulase is able to conduct the saccharification treatment sufficiently at a small amount of enzyme. Thus the chemical substances from a biomass source will be expected to have increasing popularity because it relates to a drastic cost reduction to manufacture.

### BRIEF DESCRIPTION OF THE DRAWINGS

FIG. 1 is an approximate cross section depicting the microwave radiating device of the present invention.

FIG. 2 is an approximate sketch depicting the connecting type microwave radiating device as well as related peripheral equipments of the present invention.

FIG. 3 is a partial cross section depicting the connecting type microwave radiating device of the present invention.

FIG. 4 is a partial cross section depicting a microwave radiating device which is installed at the center of one of the connecting type microwave radiating devices shown in FIG. 2.

FIG. 5 is an approximate sketch depicting a traditional microwave radiating device.

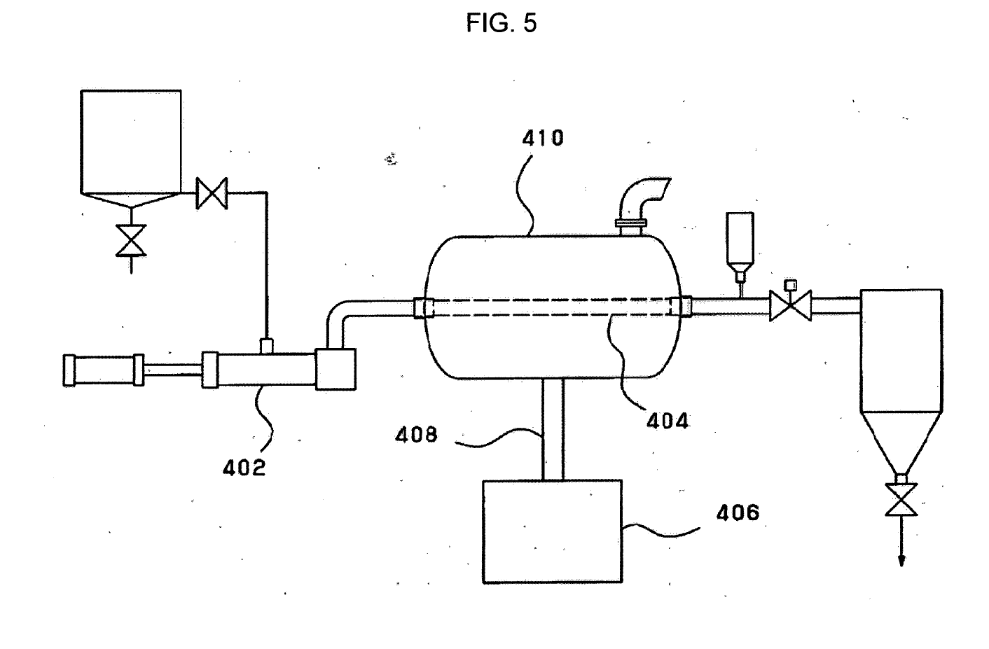

FIG. 6 is a chart depicting a temperature change of a radiated material of the example 1.

FIG. 7 is a bar chart depicting a saccharide yield corresponding to the weight of plant material of the example 8 and the comparison 2 of the present invention.

FIG. 8 is a diagram depicting results of mass analysis of the example 9 and the comparison 3 of the present invention by gas chromatography mass spectrometry.

FIG. 9 is a bar chart depicting a saccharide yield while the solid state polysaccharides obtained at the example 1 of the present invention is saccharized by enzyme.

### DETAILED DESCRIPTION OF THE INVENTION

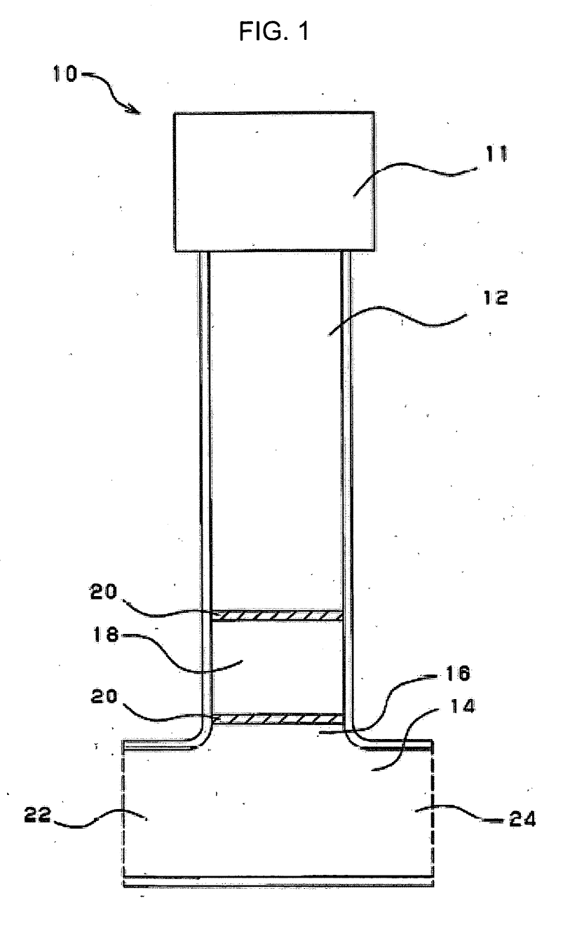

In the following, the mode of carrying out the present invention will be illustrated. The same reference number showing in the accompanying drawings below indicates the same thing. FIG. 1 is an approximate cross section depicting the microwave radiating device 10 of the present invention. Microwave radiating means consists of a microwave radiating source 11 and a waveguide 12; the waveguide 12 connects to a container 14 with a removable means. Further, the container 14 has a microwave receiving section 16 as shown in FIG. 1. The microwave receiving section 16 is shielded by dielectric material 18. Moreover, the dielectric material 18 is sandwiched by two pieces of sheet shaped parts 20. The container 14 has a supply entrance 22 as a supply means and a discharge exit 24 as discharge means.

Here the said microwave represents a short wavelength portion among electromagnetic waves; the wavelength is at a frequency of 300 MHz-30 GHz. However the said microwave is not only at the frequency described above but also at other frequencies in addition to that said frequency.

The said microwave radiating source 11 represents a substance which generates a microwave; klystron or magnetron, IMPATT diode, gun diode etc. may be used. The microwave radiating source 11 connects to the microwave receiving section 16 of the container 14 through a waveguide 12 as shown in FIG. 1, but it may connect directly to the microwave receiving section 16 of the container 14 without a waveguide 12.

The said waveguide 12 is a tube for making microwaves generated at a microwave radiating source inside. To prevent microwave from escaping to the outside, it is desirable that the waveguide 12 is made of non-dielectric material which can not be penetrated by microwaves. The materials which can be used to form the waveguide 12 are stainless steel, aluminium, titanium, nickel, gold, silver etc.

The microwave radiating means which has the microwave radiating source 11 and the waveguide 12, may connect with container 14 in a removable mode. The connecting manner may use a pressure clamp etc.

The container 14 has a microwave receiving section 16 at the wave guide direction (FIG. 1). The microwave receiving section makes the microwave penetrate into the container 14 through a dielectric material 18 as a medium. Concretely, the microwave receiving section 16 may be for example, a structure that shields the opening installed at the microwave waveguide direction of the container 14 with dielectric material 18.

The said dielectric material 18 is a material which has a property that microwaves can penetrate easily, such as quartz glass, fluorocarbon polymers, ceramics, alumina, sapphire, and diamond etc. Thus microwaves passing through the waveguide 12 will enter into the container 14 from the microwave receiving section 16 because it can penetrate the dielectric material 18. The properties or shapes of the dielectric material 18 may be chosen by the users freely to meet the purpose of using the microwave radiating device. Because the waveguide 12 and the container 14 are connected in a removable mode, it is easy to exchange the dielectric material 18 with the most suitable substance.

The dielectric material 18 is sandwiched by sheet shaped parts. The sheet shaped parts may be composed of either dielectric material or non-dielectric material. The dielectric material may use quartz glass, fluorocarbon polymers, ceramics, alumina, sapphire, and diamond etc. Here the sheet shaped parts 20 of non-dielectric material must have a structure such as a hole that is able to let microwaves penetrate. Because it is necessary to protect the dielectric material 18, it will be desirable that substances composing the sheet shaped parts 20 possess better strength than that of the dielectric material 18. The dielectric material 18 is laminated either at one side of the sheet shaped parts 20 or at both sides to make a sandwich. However, because the dielectric material 18 is protected by being sandwiched, manufacture or maintenance of the microwave radiating device becomes easy. Further, the properties or shapes of the sheet shaped parts 20 may be chosen by the users freely to meet the purpose of using the microwave radiating device. Because the waveguide 12 and the container 14 are connected in a removable mode, it is easy to exchange the sheet shaped parts 20 with the most suitable substance.

The container 14 has a structure to prevent microwaves from escaping by installing at least one layer composed of non-dielectric material between atmosphere of the inner side and the outer side of the container. Here the said non-dielectric material represents a material which has a property that microwaves can not penetrate. Materials such as stainless steel, aluminium, titanium, nickel, gold, silver etc. can be used. The outer shell of the container 14 may be made of either at least one layer of the non-dielectric material or a multilayer structure that has another layer at an inner side or an outer side atmosphere of the layer of the non-dielectric material. Further, the surface of the outer shell of the container 14 which is composed of dielectric material may be coated with a layer of non-dielectric material.

Microwaves penetrate the microwave receiving section 16 of the dielectric material 18 and enters the container 14, when microwaves contact a material it is absorbed by the material. Further, microwaves that are not absorbed by the radiated material are reflected at the inner wall of the container 14 and moved inside of the container 14. Because the container 14 has installed a layer composed of non-dielectric material as described above, microwaves can not escape to the outside of the container and decrease energy loss.

The container 14 has a supply entrance 22 as a supply means and a discharge exit 24 as discharge means. The radiated material is given into the container 14 from the supply entrance 22 by a pump pressure, and discharged to outside of the container 14 from the discharge exit 24 after the irradiation of microwaves. Further, the numbers of supply entrances 22 and the discharge exits 24 have no limit and can be prepared to meet the objects of usage. For example, at the condition that more than two kinds of radiated materials are mixed to use, the number of supply entrances 22 may be the same as the number of the kinds of the radiated materials, and the ratio of the mixed amount of the radiated materials can be managed by the supplying amount. Further, it may be desired that the discharge exit 24 also has a plural number to respond to treatment after irradiation.

The connecting type microwave radiating device according to the present invention is a combination device that combines a microwave radiating device 10 mentioned above with removable means. The removable means may use a pressure clamp etc. Further, the number of connecting microwave radiating devices can be established freely to meet a user's demands of a microwave treatment amount.

In the connecting type microwave radiating device, when microwave irradiates from multiple microwave radiating sources 11, it is desirable that the dielectric material 18 which shields the microwave radiating source 11 or microwave receiving section is optimized for preventing microwaves from counteracting each other.

The microwave radiating device and the connecting type microwave radiating device according to the present invention have various usages, for example, enhancement and high yield rate of a chemical reaction, pretreatment of biomass raw material such as grain residue or disused wood etc., sterilization of food or medicine, extraction or dehydration of a substance etc.

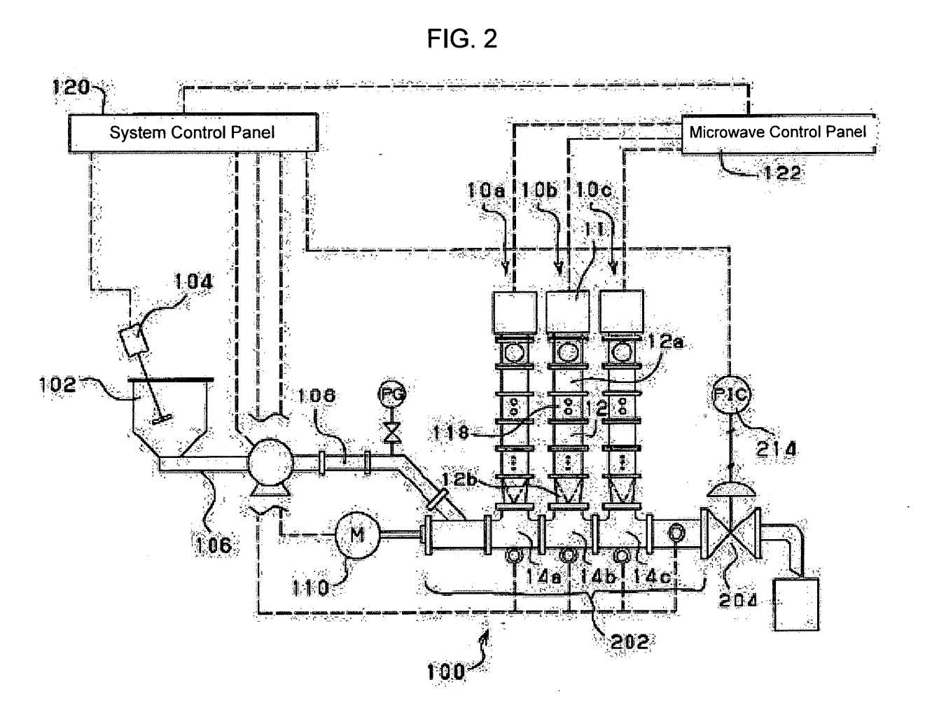

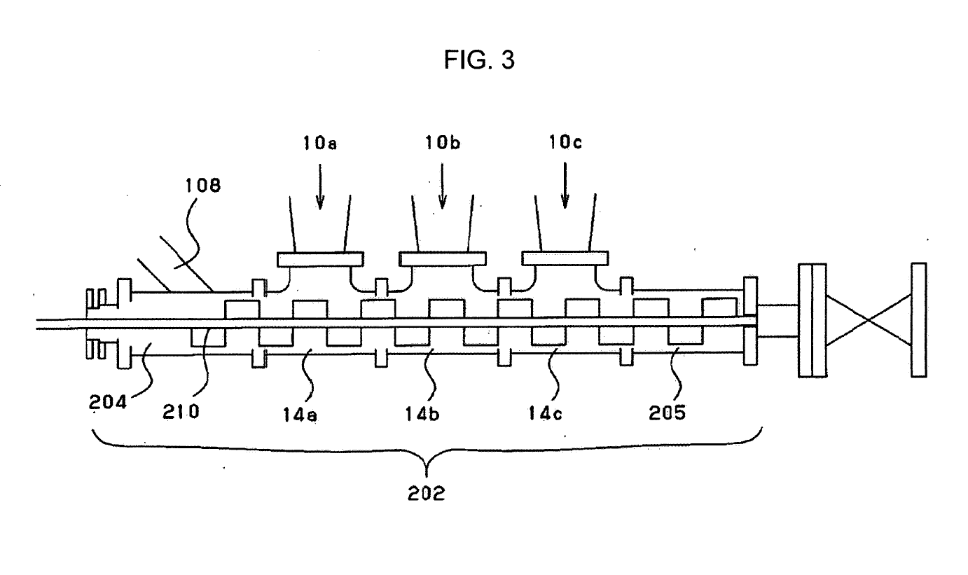

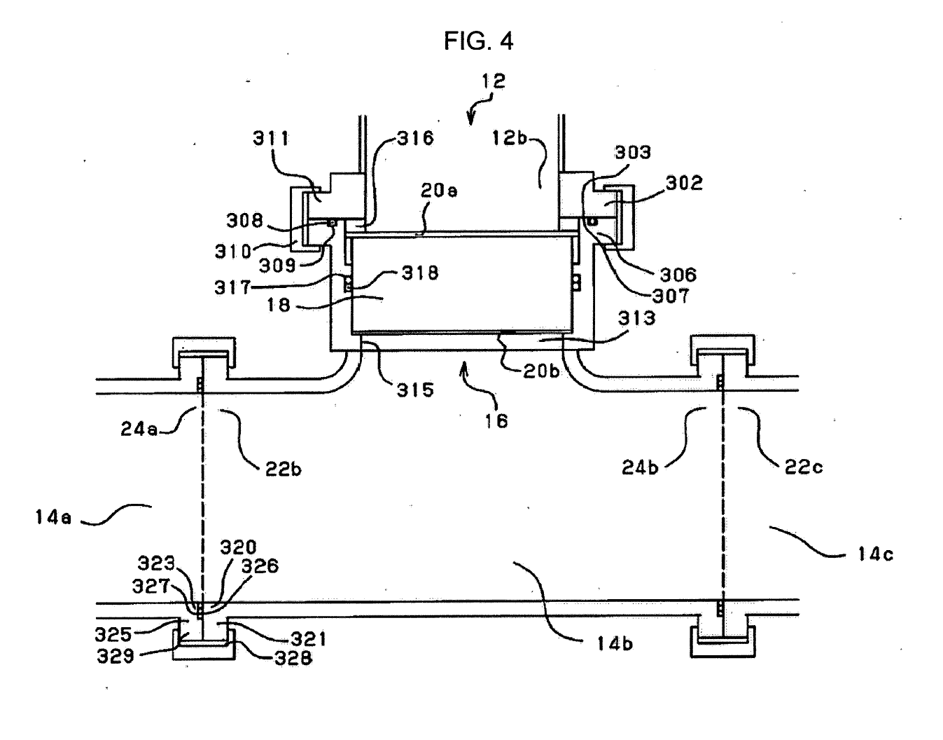

In the following one example, the microwave radiating device and the connecting type microwave radiating device according to the present invention will be described in detail with referring to FIG. 2, FIG. 3, and FIG. 4. FIG. 2 depicts the connecting type microwave radiating device 100 of the present invention and related peripheral equipment. This connecting type microwave radiating device 100 is a combination device wherein three microwave radiating devices of the present invention, e.g. 10a, 10b, and 10c, are connected. Further, FIG. 3 is a partial cross section (view direction is perpendicular to the paper) depicting the above connecting type microwave radiating device 100. Further, FIG. 4 is a partial cross section (view direction is perpendicular to the paper) depicting a microwave radiating device which is installed at the center of the above connecting type microwave radiating device 100.

Now referring to FIG. 2, the composition of the corresponding portion of microwave radiating means will be described. The microwave radiating source 11 connects to one end 12a of the waveguide 12. The other end 12b of the waveguide 12 connects to container 14b. Further, a power monitor 118 is installed in the waveguide 12 to measure the electricity of microwaves that enter into the waveguide 12.

Here the connection portion of the waveguide 12 and the container 14b will be described with referring to FIG. 4. The waveguide 12 has an edge 302 at container brim 12b. The edge 302 protrudes toward a diameter direction, and forms a unity as a portion of the waveguide 12. On the other hand, the container 14b also has an edge 306 at upper side of the microwave receiving section 16. The edge 306 is also protruding towards the diameter direction. Here the edge 302 of the waveguide 12 has a conjugating section 303, and the edge 306 of the container 14b also has a conjugating section 307, and both edges are able to conjugate to each other. Further, a gap 309 is provided at conjugating section 307 of the edge 306 of the container 14b, annular elastic parts 308 such as an O-ring is inserted into the gap 309. By doing so the small aperture formed by the edge 302 and the edge 306 conjugate together and are filled up; and microwaves are prevented from escaping to the outside of the device. Further, when the edge 302 and the edge 306 conjugate to each other, the pressure clamp 310 is inserted into the outside protruding section 311 of the edges. The result is that the edges 302 and 306 join together in up and down directions, so the waveguide 12 and the container 14b are fixed. Further, because the pressure clamp 310 is dismantled easily the connection of the waveguide 12 and the container 14b is removable.

The microwave receiving section 16 of the container 14b is shielded by glass 18 which is a dielectric material. The container 14b has a glass housing section 313, and the glass 18 is kept in the glass housing section 313. Furthermore, glass 18 is sandwiched by gasket 20a, and 20b which are sheet shaped parts composed of polytetrafluoroethylene. Here because the microwave receiving section 16 has a protruding section 315, the diameter of the microwave receiving section 16 is smaller than that of the glass 18 and the gasket 20a, and 20b. Therefore the protruding section 315 bears the glass 18 and the gasket 20a, and 20b. The result is that the glass 18 and the gasket 20a, and 20b will not fall into the container 14b.

Further, the gasket 20a at the upper side of the glass 18, contacts a spacer 316. The pressure that makes the waveguide 12 and the container 14b fixed is transmitted to the upper gasket 20a, glass 18, and lower gasket 20b through the spacer 316. Meanwhile, because the lower gasket 20b contacts protruding section 315, a counter force to above pressure is transmitted to the lower gasket 20b, glass 18, and upper gasket 20a. Thus glass 18, and gasket 20a, 20b are also fixed in the up and down directions without using special conjugating means. Further, microwave treatment should be able to endure the pressure from the container 14b or waveguide 12, to be implemented even under the severe conditions such as high pressure or decompression, vacuum etc. Further, because the gasket 20a, 20b have better strength than that of the glass 18, the glass 18 is protected by the gasket 20a, 20b.

Further, in the inner wall of glass housing section 313 there is installed a gap 317 at a peripheral direction. An annular elastic part is inserted into the gap 317 to prevent microwaves from escaping.

The container 14b provides one supply entrance 22b and one discharge exit 24b, respectively. The supply entrance 22b has an edge 321. The shape of the edge 321 protrudes toward the diameter direction. Meanwhile, as shown in FIG. 4 the discharge exit 24a of the microwave radiating device 10a at left side also has an edge 325. The shape of the edge 325 protrudes toward the diameter direction, too. Here the edge 321 at the supply entrance 22b side has a conjugating section 320, the edge 325 at the discharge exit 24a has a conjugating section 323, and both edges are conjugating sections and are able to conjugate to each other. Further, a gap 327 is provided at the conjugating section 323 of the edge 325 of the discharge exit 24a, an annular elastic part 326 such as an O-ring is inserted into the gap 326. By doing so the small aperture caused during the edge 321 and the edge 325 conjugate together, and is filled up; and microwaves and radiated material are prevented from escaping to the outside of the container 14a, 14b. Further, when the edge 302 and the edge 306 conjugate to each other, the pressure clamp 328 is inserted from outside into the outer protruding section 329 of the edges. The result is that the edge 321 and 325 join together in right and left directions, so the container 14a and the container 14b are fixed. Further, because the pressure clamp 328 is dismantled easily, the connection of the container 14a and the container 14b is removable.

The discharge exit 24b connects to the supply entrance 22c of the microwave radiating device 10c shownin FIG. 4. Namely, the discharge exit 24b is equivalent to the discharge exit 24a of the microwave radiating device 10a, and the supply entrance 22c is equivalent to the supply entrance 22b of the microwave radiating device 10b. Therefore the discharge exit 24b and the supply entrance 22c may connect in a removable mode by the same connecting manner as the discharge exit 24a and the supply entrance 22b mentioned above.

Referring to FIG. 2, the structure of the radiated material supply will be described in detail below. The radiated material is thrown into a raw material tank 102, and mixed uniformly by a raw material mixer 104, then delivered to a supply pump 106. The supply pump 106 pressurizes to push the radiated material out. Then the radiated material delivered to the pump 106 goes through a supply tube 108, and reaches the internal space of a reacting tube 202.

Referring to FIG. 3, the step relating to the material irradiated by microwaves will be described in detail below. The reacting tube 202 is composed of an anterior reacting chamber 204, and three containers of the microwave radiating device 14a, 14b, 14c, and a posterior reacting chamber 205. The radiated material that has gone through the supply tube 108 reaches the anterior reacting chamber 204. A reacting stirring stick 210 is installed along the axis direction of the interior of the reacting tube 202. Multiple impellers are mounted on this reacting tube 202 along the axis of the tube. These impellers will display a screw effect during the time that the reacting stirring stick 210 rotates. Therefore when the reacting stirring stick 210 is rotated by a motor of a reacting mixer 110, the radiated material is mixed uniformly, while delivered from the left side to the right side as shown in FIG. 3. Microwave irradiates from microwave radiating means of three microwave radiating devices 10a, 10b, 10c. In the container 14a, 14b, and 14c, there are provided thermometers to manage the temperature of the interior of the reacting tube 202. The radiated material that had been irradiated by microwaves is delivered to posterior reacting chamber 205.

Referring to FIG. 2 again, the step relating to the radiated material that is discharged from a connecting type microwave radiating device 100 will be described in detail below. The radiated material that is delivered to the posterior reacting chamber 205 is discharged to outside of the device from a control valve 204. At the control valve 204 there is joined a pressure adjuster 214 to adjust the internal air pressure of the reacting tube 202.

Referring to FIG. 2, an outline of the whole control of the connecting type microwave radiating device is given below. The control of the connecting type microwave radiating device is undertaken by a system control panel 120 and a microwave control panel 122. The system control panel 120 executes control of the raw material agitator 104, the supply pump 106, the reacting mixer 110, and the pressure adjuster 214. The internal temperature of the reacting tube is measured by the thermometer, and transmitted to the system control panel 120. Further, the microwave control panel 122 executes control of the microwave radiating source 11. The measured result of power-monitor 118 is transmitted to the microwave control panel 122. The system control panel 120 and the microwave control panel 122 exchange data information with each other, and compel the connecting type microwave radiating device to work appropriately.

The mode of carrying out the microwave radiating device, and the connecting type microwave radiating device according to the present invention has been described above, but the mode described here does not exceed one example of the present invention. Although certain illustrative embodiments and methods have been disclosed herein, it will be apparent from the foregoing disclosure to those skilled in the art that variations and modifications of such embodiments and methods may be made without departing from the spirit and scope of the invention. For example, the connecting type microwave radiating device can be either that composed of more than three of microwave radiating devices, or that microwave radiating source and container directly joined without using a waveguide in each microwave radiating device.

Next the method relating to producing a sugar ingredient from plant material will be described. The method comprises a separation process which obtains a mixture through microwaves radiating on plant material which contains lignocellulose with the existence of a Lewis acid catalyst; the mixture contains decomposed products of lignin and sugar ingredients that contain solid state polysaccharides; and a separation process which obtains a mixture through microwaves radiating on plant material which contains lignocellulose with the existence of molybdic acid ion, ammonium ion and peroxides, the mixture contains decomposed products of lignin and sugar ingredient that contains solid state polysaccharides.

The background technique is now described.

Recently the problems about the deterioration of the earth environment and the exhaustion of fossil resources are arising, and continuous recycling is bringing attention to biomass as a new resource. Especially the concept of a “biorefinery” which means that chemical products or energy are obtained from renewable biomass, which is becoming a most urgent task to realize.

At the present grains are used as a biomass resource, but popularity on a large scale can not be achieved because of the high cost and the rising grain price. Therefore, the inventors are dedicated to the research and development of using lignocellulose which is contained in plant material. The said lignocellulose represents botanical organic compounds which are composed of polysaccharides such as cellulose, hemicellulose etc. Because the polysaccharides such as cellulose, hemicellulose etc. are used as a resource, to separate saccharides and lignin, namely to destroy the insulation of lignin over polysaccharides, has proven effective. Particularly if lignocellulose is made to convert to useful substances such as ethanol etc. by enzymatic saccharification treatment and fermentation, the pretreatment that destroys the insulation of lignin over polysaccharides is necessary.

There are developing various methods concerning the production of a sugar ingredient by separating saccharides existing in lignocellulose and lignin. For example, there are published: the method of depolymerization of lignin in water solution containing copper and peroxide; the method of depolymerization of lignin in water solution containing molybdic acid or peroxide; the method of decomposition of lignin of ligneous biomass by hydrogen peroxide water solution which contains a very small amount of tungsten oxide or molybdate catalyst; the method of enhancing saccharification reaction by using sodium tungstate (Na₂WO₄) or Disodium molybdate (Na₂MoO₄) to remove the lignin contained in old paper; the method of hastening de-lignin by combining organic solvent and Lewis acid etc.

Further, there are also developing various methods concerning the production of sugar ingredient through internal heating by microwave irradiating in addition to external heating. There are disclosed: the method of conducting pretreatment by means of microwave irradiating in water solution of acetic acid (non-Patent document 2); the method of conducting separation, decomposition of lignin by implement microwave irradiating at existence of oxidant (Patent document 5); the method of accelerating the speed of de-lignin during digestion (Patent document 6); the method of combining microwave treatment and alkaline treatment (non-Patent document 3) etc.

However, according to the traditional method of producing a sugar ingredient by microwave irradiation or external heating, the temperature that separation of saccharides and lignin occurs, is near the temperature of over-decomposition of saccharides; the problem of yielding large amount of over-decomposition products happens. Further, if the saccharides are over-decomposed, furfurals such as 5-hydroxymethyl furfural or furfural will be formed, these furfurals will hinder the fermentation of alcohol from saccharides. Therefore the method of external heating is not suitable for a biorefinery.

Further, the lignin of a conifer is hard to decompose. In a traditional method that uses microwave radiation, decomposition of the lignin of the conifer is not sufficient, the amount of obtained sugar ingredient is very small. On the other hand, as to herbs or broadleaf trees material the treatment temperature is over 200° C. by the traditional method, the problems are huge energy consumption and large amount of over-decomposed products of saccharides.

Further, the useful substances such as bioethanol etc. obtained from biomass are too expensive to be popular, therefore a plan to take the cost down is necessary. For getting ethanol from biomass it is necessary to conduct enzymatic saccharification treatment towards polysaccharides contained in the produced sugar ingredient. But in the traditional method large amount of enzyme is used to secure the producing amount of saccharides. Therefore if there is a method to decrease the amount of enzyme for saccharification treatment, it will be able to cut the cost down largely.

Further, in the decomposition process of lignin, if organic substances such as propanol or butanol etc. are used as a dispersing medium, great labor is needed in post-treatment. Further, if enzymatic saccharification treatment is conducted after the decomposition process of lignin, it is necessary to remove organic substances. Therefore a method of using water as a dispersing medium is desired.

Moreover, for decreasing the environment impact, it is requested a reaction system at conditions of low temperature or without using any harmful substances such as sulfuric acid or phenol.

Namely, the objects of the present invention are to provide a method of producing a sugar ingredient from a plant material: the method being able to produce the sugar ingredient efficiently meanwhile preventing over-decomposition of saccharides in a condition of a low environment impact; to obtain polysaccharides by sufficient saccharification under the condition of using a small amount of enzyme.

Further, the objects of the present invention are to provide a method of producing a sugar ingredient from a plant material: the method being able to produce sugar ingredient efficiently meanwhile preventing over-decomposition of saccharides in condition of a low environment impact; to obtain polysaccharides by sufficient saccharification under a condition of using water as a dispersing medium or using a small amount of enzyme.

In the following, the mode of carrying out the present invention will be described.

The present invention is related to a method of producing a sugar ingredient from a plant material: the method comprises a separation process which obtains a mixture through microwave radiating on plant material with the existence of a Lewis acid catalyst; the plant material at least contains lignocelluloses; the mixture contains decomposed products of lignin and a sugar ingredient that at least contains solid state polysaccharides. Further, the present invention is related to a method of producing a sugar ingredient from a plant material: the method comprises a separation process which obtains a mixture through microwave radiating on plant material with the existence of a molybdic acid ion, ammonium ion and peroxides; the plant material at least contains lignocelluloses; the mixture contains decomposed products of lignin and a sugar ingredient that at least contains solid state polysaccharides. Namely, the method of producing a sugar ingredient from a plant material according to the present invention is characterized by molecule diminishment of lignon in lignocellulose, decoupling of the linkage between lignin and polysaccharides, change of the structure of polysaccharide crystal, leaching of hemicellulose etc.

In the separation process of the present invention, the sugar ingredient and the lignin are separated through molecule diminishment of lignon in lignocellulose, decoupling of the linkage between lignin and polysaccharides, change of the structure of polysaccharide crystal, leaching of hemicellulose etc. Therefore the mixture obtained by this process comprises a sugar ingredient which contains a monosaccharide or a solid state polysaccharides such as cellulose etc., and decomposition products of lignin in a dissolution condition. Hence it does not matter that whether or not, the method of producing the sugar ingredient from a plant material according to the present invention further comprises an extraction process to extract the above solid state polysaccharides obtained by using biomass from the above mixture.

In the following a favorable embodiment of the method of producing a sugar ingredient from a plant material according to the present invention comprises the above separation process and extraction process, will be described, but the present invention is not limited to those described here.

### (1) Separation Process

In the method of producing a sugar ingredient from a plant material according to the present invention, a mixture, that contains decomposed products of lignin and a sugar ingredient which at least has solid state polysaccharides, is obtained; by means of an irradiating microwave in the existence of a Lewis acid to plant material that has at least lignocellulose.

Further, the method of producing a sugar ingredient from a plant material according to the present invention, a mixture, that contains decomposed products of lignin and a sugar ingredient which at least has solid state polysaccharides, is obtained; by means of at the existence of molybdic acid ion, ammonium ion and peroxides irradiating microwave to plant material which has at least lignocellulose.

The mixture obtained from the process comprises decomposed products of lignin and a sugar ingredient which has at least solid state polysaccharides. The decomposed products of lignin comprise aromatic compounds such as phenol etc. Further, the sugar ingredient which has at least solid state polysaccharides, consists of low molecule saccharides such as monosaccharides or oligosaccharides etc. in addition to solid state polysaccharides such as cellulose, hemicellulose etc.

The solid state polysaccharides contained in the sugar ingredient are converted to low molecule saccharides by conducting saccharification treatment. Further, the invention is able to produce useful chemical substances such as ethanol, methane, succinic acid, itaconic acid, lactic acid etc. by conducting fermentation treatment etc. Further, it is also possible to produce useful chemical substances from the low molecule saccharides contained in the sugar ingredient or the decomposed products of lignin.

The irradiation of microwave in this process is a mode of heating wherein radiation causes oscillation effect of an electromagnetic field on a charged particle or electric dipole inside of a substance of plant material; makes the particle rotate or oscillate, then self-heat from the interior. This mode the heating is different from that of an external heating source, it is able to heat the plant material rapidly and uniformly, and almost ignores the effects of thermal conduction or convection.

In this step microwave represents a short wave length portion of the electromagnetic wave, comprising an electromagnetic wave with a wave length less than 1 m or a submillimetric wave with a wave length less than 1 mm which is close to far infrared. The ideal frequency of the microwave of the present invention is 300 MHz-30 GHz, with a more ideal frequency of the microwave is 2000 MHz-6000 MHZ. Moreover, microwave with frequency 2450 MHz or microwave with frequency 5800 MHz are used often. However, microwave used in this process is not limited to the range of these frequencies, microwave beyond the range of these frequencies is used also. As described above when microwave irradiates a material, the radiated material oscillates responding to the microwave, then the material generates heat itself because of frictional heat occurring by oscillation. Although the effect of the present invention can be obtained in the range mentioned above, the most favorable mode is to optimize wave length and frequency of the microwave in response to the growing area, structure, and kinds etc. of the plant material as well as the method of pretreatment.

The plant material used as raw material in this process represents a material of a botanical source comprising at least lignocellulose, further compring lignin, cellulose, hemicellulose. The plant material is for example, a material of wood source or herb source etc. The plant material of wood source may use a gymnosperm comprising a conifer such as cryptomeria, pine, Japanese cypress, buddhist pine, Cephalotaxus, Californian redwood, hiba arborvitae, Japanese yew etc., or ginkgo; an angiosperm comprising broad leaf trees such as beech tree, Japanese zelkova keyaki tree, camellia, Japanese oak, cherry tree, camphor tree, Castanopsis, maple tree, chestnut tree, eucalyptus, a locust tree etc. Furthermore, the lignin that contained in the plant material of the conifer source is mainly constituted of guaiacyllignin, while the lignin that is contained in the plant material of a broad leaf tree source is mainly constituted of guaiacyllignin and syringyllignin. The plant material of a herb source may use bagasse which is a residue of Saccharum officinarum, rice straw, wheat straw, rice husks etc.

The plant material used as a raw material in this process may be managed by the methods well-known in the art and used in the form of a powder or chip, flake, fiber, lumber etc. From the point of view about reaction efficiency the favorable plant material is manufactured to powder, chip, flake, or fiber.

The diameter of the particles during processing a plant material to particle is not limited, but favorable diameter would be 0.1 μm-5 mm. Further, the size or thickness is not limited during processing a plant material to chip, but favorable size is 5 mm-50 mm, and favorable thickness is 0.1 mm-5 mm.

Further, for the efficiency of producing a sugar ingredient, it is favorable to implement adjustment by pretreatment which removes hydrophobic low molecules through refluxing a mixed solution of alcohol and benzene; or removes hydrophilic low molecules through mixing with water and heating. The above pretreatment may be omitted if high yield is secured at the saccharification treatment. After pretreatment the plant material may be used in this process either being dehydrated or being not dehydrated.

The Lewis acid catalyst used in this process represents a catalyst which has activity to receive electron-pair from reaction partner, excludes usual acid catalyst (inorganic acid such as hydrochloric acid, sulfuric acid etc. or organic acid such as phosphoric acid). The Lewis acid may use metallic salt, halogenides of metal, or oxides of metal, carboxylates etc. To be concrete, aluminium compounds such as aluminium floride, aluminium chloride, aluminium bromide, aluminium iodide, aluminium sulfate etc.; copper compounds such as copper(I) fluoride, copper(II) fluoride, copper(I) chloride, copper(II) chloride, copper(I) bromide, copper(II) bromide, copper(I) iodide, copper(II) iodide, copper(I) sulfate, copper(II) sulfate etc.; iron compounds such as ferrous(Iron(II)) floride, ferric(Iron(III)) floride, ferrous chloride, ferric chloride, ferrous bromide, ferric bromide, ferrous iodide, ferric iodide, ferrous sulfate, ferric sulfate etc.; zinc compounds such as zinc floride, zinc chloride, zinc bromide, zinc iodide, zinc sulfate etc.; silver compounds such as silver floride, silver chloride, silver bromide, silver iodide etc.; boron compounds such as boron floride, boron chloride etc.; titanium compounds such as titanium(IV) chloride etc.; nickel compounds such as nickel chloride etc.; scandium compounds such as scandium trifluoromethanesulfonic acid etc.; palladium compounds; vanadium compounds are used, but not limited to these compounds.

Among those compounds above, for the reason of low environment impact due to easy management of liquid wastes, and low cost for handling, it is favorable to use at least one selected from the groups consists of aluminium chloride, aluminium bromide, aluminium sulfate, ferric chloride, zinc bromide, and zinc sulfate.

The compound which may be used as a dispersing medium in this process is, for example, water or organic compounds. Concretely, compounds used as examples of the organic compounds comprise glycerin, ethylene glycol, propylene glycol, methanol, ethanol, propanol, butanol, octanol, butandiol, trimethylolpropane, methylethyl ketone, acetylacetone, dimethyl sulfoxide, nitrobenzene etc. Either using water or organic compounds singly, or using a mixture of water and organic compounds will be proper. In the condition of using a mixture of water and organic compound it is favorable that 1 volume of water is mixed with 0.1 volume-10 volume of the organic compounds. Further, it is also favorable that the dispersing medium contains acid or alkali.

Among those compounds above, from the point of view of not giving loading to the environment it is favorable to use a mixture of propanol and water. Further, from the point of view of decreasing the environment impact it is favorable to use a compound which converts microwave energy to heat in high efficiency. The efficiency of converting microwave energy to heat is a quotient wherein dielectric loss is divided by relative permittivity, and is expressed as a numerical value of loss angle (tangent delta). The examples of organic compounds with large loss angle will be given as ethylene glycol, ethanol, dimethyl sulfoxide, propanol, methanol, butanol etc., and these compounds may be heated with little energy by being used singly or mixed with water.

When microwave irradiates to the plant material in the presence of Lewis acid, for example, microwave may irradiate to the above plant material through the way that microwave irradiates to plant material in a dispersing liquid in which powder, chips of plant material are dispersed in the above dispersing medium. But sometimes a partial portion of the plant material may dissolve in a dispersing medium. It is acceptable that either a Lewis acid catalyst is previously dissolved in the dispersing medium before the plant material is dispersed, or is added into the dispersing medium after the plant material has been dispersed.

The ratio by weight of the amount of plant material and the amount of dispersing medium is not limited. The ideal ratio by weight of plant material vs. dispersing medium is 1:1-1:100, a more ideal ratio by weight may be plant material vs. dispersing medium of 1:5-1:20. If the amount of dispersing medium is small, the heating rate may slow down; on the other hand, if the amount of solvent is large, the amount of plant material for treatment becomes small, the producing cost becomes expensive.

The optimized amount of the Lewis acid catalyst is varied according to the variety of plant material. For example, if the source of plant material is a beech tree, corresponding to 1 g of plant material, the ideal amount of Lewis acid catalyst is 1 μmol-1000 μmol, more ideal amount is 60 μmol-720 μmol, yet a more ideal amount is 180 μmol-360 μmol. If the amount of Lewis acid corresponding to plant material is insufficient, a satisfying effect of separating saccharides and lignin may not be obtained; on the other hand, if the amount of Lewis acid corresponding to plant material is too much, the treatment of solvent becomes difficult after reaction.

When microwave irradiates to the plant material in the above dispersing medium according to this process, it is favorable that the dispersing liquid is stirred by using a stirrer to allow the microwave to radiate uniformly to the plant material which is dispersed in the above dispersing medium.

The reaction time of implementing microwave radiation of this process has no limitation, but 1 minute to 60 minutes will be favorable. If the reaction time is short, the separation of saccharides and lignin will not be sufficient; on the other hand, if the reaction time is long, not only the cost is high but also the useful chemical substances are possibly decomposed.

The reaction temperature of implementing separation of saccharides and lignin of this process has no limitation, but to heat the plant material at 80° C.-240° C. will be favorable, and to heat the plant material at 150° C.-180° C. will be more favorable. If the reaction temperature is low, the separation of saccharides and lignin would not be sufficient; on the other hand, if the reaction temperature is high, the useful chemical substances are possibly decomposed. In accordance with the present invention, at a temperature that saccharides will not over-decompose, it is able to separate saccharides and lignin efficiently. Further, in the reaction of the present invention it is favorable to achieve a prescribed reaction temperature within a short time by rapidly heating. If the heating is rapid, by-products produced at a low temperature are rare.

The ideal molybdic acid ion used in this process is an ion comprised of molybdenum atoms and oxygen atoms,such as, compounds expressed with a chemical formula MoO₄²⁻, Mo₂O₄²⁻, Mo₆O₁₉²⁻, Mo₇O₂₄⁶⁻, are given as examples. Further, the ammonium ion used in this process is expressed with a chemical formula NH₄⁺.

In this process microwave irradiates in the presence of the above molybdic acid ion, the above ammonium ion, and peroxides. The above two ions are obtained by dissolving molybdate salt and ammonium salt in a dispersing medium which is described later. It is favorable that when dissolving ammonium molybdate the above two ions are able to obtain simultaneously. It is acceptable that either those salts are previously dissolved in a dispersing medium before the plant material is dispersed, or are added into the dispersing medium and dissolved after the plant material has been dispersed.

In this process the peroxides may use hydrogen peroxide or metal salt of hydrogen peroxide, organic compounds having peroxy group etc. Metal salt of hydrogen peroxide such as lithium peroxide, potassium peroxide, sodium peroxide, magnesium peroxide, calcium peroxide, barium peroxide etc. are given as examples. Among these peroxides, from the point of view of decreasing cost, it is favorable to use hydrogen peroxide. It is acceptable that either those peroxides are previously dissolved in the dispersing medium before the plant material is dispersed, or are added into the dispersing medium and dissolved or mixed after the plant material has been dispersed. Further, it is also favorable to generate peroxide in the dispersing medium by using well-known methods in the art such as the anthraquinone method.

The dispersing medium which may be used in this process, for instance, may be water, organic compounds, or their combination. The organic compounds such as glycerin, ethylene glycol, propylene glycol, methanol, ethanol, propanol, butanol, octanol, butandiol, trimethyloloropane, methylethyl ketone, acetyl acetone, dimethylsulfoxide, nitrobenzene etc. are given as examples. Among these compounds from the point of view of decreasing trouble of post-treatment and the cost, it is favorable to use water as the dispersing medium. Further, it is also favorable that the dispersing medium contains acid or alkali.

When microwave irradiates to the plant material at the existence of molybdic acid ion, ammonium ion, and peroxides, for example, microwave may irradiate to the above plant material through the way that microwave irradiates to plant material in a dispersing liquid wherein powder, chip of plant material are dispersed in above dispersing medium. But sometimes a partial portion of the plant material may dissolve in the dispersing medium.

The optimized concentration of molybdic acid ion, ammonium ion, and peroxide is varied according to the variety of plant material. The ideal final concentration of molybdic acid ion and ammonium ion vs. plant material such as a beech tree is 0.01 mM-100 mM; a more ideal concentration is 0.1 mM-10 mM; yet a more ideal concentration is 0.25 mM-5 mM. Further, the ideal final concentration of peroxide vs. plant material such as beech tree is 0.01M-10M; a more ideal concentration is 0.1M-1M. If their concentration is low, the separation of saccharides and lignin will not be sufficient; on the other hand, if their concentration is high, the treatment of solvent becomes difficult after reaction.

The ratio by weight of the amount of plant material and the amount of dispersing medium in the above dispersing medium is not limited. The ideal ratio by weight of plant material vs. dispersing medium is 1:1-1:100, a more ideal ratio by weight of plant material vs. dispersing medium is 1:5-1:20. If the amount of dispersing medium is small, the heating rate may slow down; on the other hand, if the amount of solvent is large, the amount of plant material for treatment may be small, the production cost becomes expensive.

When microwave irradiates the plant material in the above dispersing medium according to this process, it is favorable that the dispersing liquid is stirred by using a stirrer to secure that microwave radiates uniformly to the plant material which is dispersed in the above dispersing medium.

The reaction time of implementing microwave radiation of this process has no limitation, but 1 minute to 60 minutes will be favorable. If the reaction time is short, the separation of saccharides and lignin will not be sufficient; on the other hand, if the reaction time is long, not only the cost is high but also the useful chemical substances are possibly decomposed.

The reaction temperature of implementing the separation of saccharides and lignin of this process has no limitation, but to heat the plant material at 50° C.-240° C. will be favorable, and to heat the plant material at 80° C.-180° C. will be more favorable. If the reaction temperature is low, the separation of saccharides and lignin would not be sufficient; on the other hand, if the reaction temperature is high, the useful chemical substances are possibly decomposed. In accordance with the present invention, at a temperature that saccharides will not over-decompose, it is able to separate saccharides and lignin efficiently. Further, in the reaction of the present invention it is favorable to achieve a prescribed reaction temperature within a short time by rapidly heating. If heating is rapid, by-products produced at low temperature are rare.

### (2) Extraction Process

Further, the present invention comprises an extraction process in which solid state polysaccharides are extracted from the mixture obtained in separation process (1). As described above the mixture obtained in separation process (1) comprises decomposed products of lignin that are dissolved in a dispersing medium, and solid state polysaccharides that are composed of cellulose, hemicellulose etc. According to this process it is able to extract an insoluble component of solid state polysaccharides that are composed of cellulose, hemicellulose etc. In the insoluble component except solid state polysaccharides, there may remain lignin which is not decomposed in the above separation process. Moreover, decomposed products of lignin contained in the above mixture or low molecule saccharides obtained from sugar ingredient are also used to produce useful chemical substances.

The solid state polysaccharides are able to be extracted by using techniques of filtration etc. in this process. The mixture obtained in separation process (1) is directed through a porous filter medium; and the solid state polysaccharides which can not pass porosity are extracted from a dispersing medium. The filter medium may use filter paper, glass fiber filter, membrane filter, filtration plate etc.

The favorable filter medium that can be used in this process is favorable to select a suitable diameter of porosity to catch obtained solid state polysaccharides reliably. For example, a filter medium that holds particles of 1 μm-50 μm is used. Further, for raising the filtration rate it is also favorable to conduct a pressure filtration such as decompression, pressurization, centrifugation etc.

The solid state polysaccharides extracted in this process are able to convert to low molecule saccharides through saccharification treatment in which an enzyme such as cellulase may be used. By using microbes to perform fermentation treatment it is able to obtain useful compounds such as ethanol, methane, succinic acid, itaconic acid, lactic acid etc. from the obtained low molecule saccharides. Further, it is able to obtain useful chemical substances such as aromatic compounds from decomposed products of lignin which are contained in the residue mixture after extraction of solid state polysaccharides.

According to this process, the solid state polysaccharides are extracted, and when the solid state polysaccharides and the decomposed products of lignin are mixed together the saccharification treatment of the solid state polysaccharides may still be conducted. In this state the saccharizied mixture obtained from saccharification treatment may have decomposed products of lignin and low molecule saccharides. As a result, the mixture may be used as raw material either in the condition of mixture, or after separating the decomposed products of lignin and the low molecule saccharides respectively by chromatography or other methods.

### EXAMPLES

On the basis of the treatment of radiated material which is utilized in the microwave radiating device of the present invention, the present invention will be described further, but specification and examples are to be regarded in an illustrative rather than a restrictive sense, and all modifications are intended to be included within the scope of the present invention.

### Example 1

The treatment of radiated material is conducted by using a connecting type microwave radiating device in which three microwave radiating devices are connected as shown in FIG. 2. The frequency of the microwave is 2450 MHz; output of the microwave radiating device 10a is 1.2 kW; output of the microwave radiating device 10b is 0.9 kW; output of the microwave radiating device 10c is 0.8 kW.

The radiated material is powder of cryptomeria which has passed through a sieve of 48 mesh. Further, the solvent is a mixture of ethylene glycol and phosphoric acid at a ratio 95:5 by volume. The powder of cryptomeria and the solvent which is 11 times of weight of the powder of cryptomeria are mixed uniformly in the raw material tank 102. The radiated material is sent into the supply pump 106, then goes through the supply tube 108 by pressurizing the supply pump 106, and achieves the interior of the reacting tube 202. The pressure of the interior of the reacting tube 202 is 1.5 MPa due to pressurizing the supply pump 106.

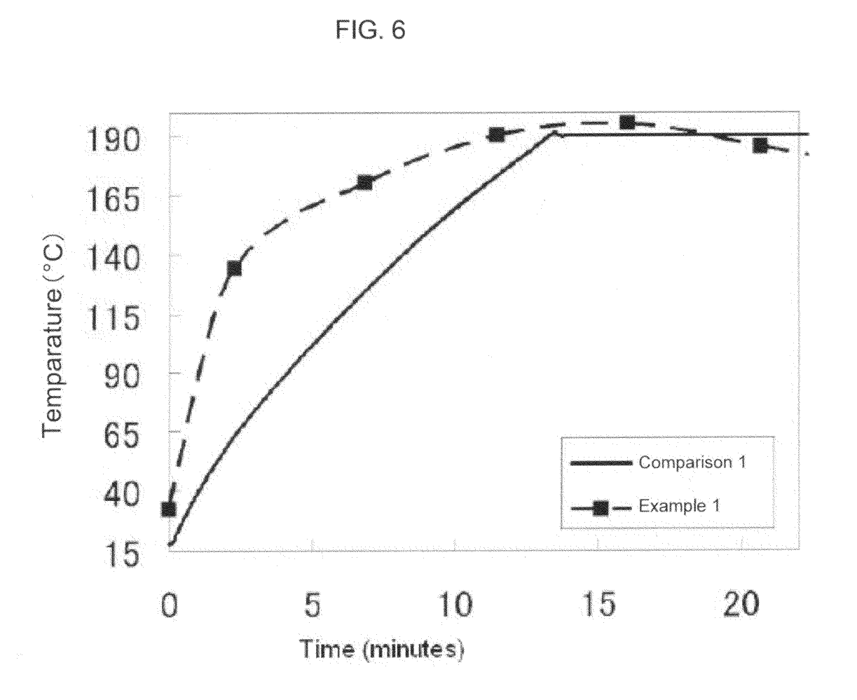

The radiated material which is sent to the anterior reacting chamber 204, is transported into the reacting tube 202 meanwhile receiving microwave irradiation treatment. Once the radiated material is sent into the supply pump 106, until it is discharged out from control valve 204, the time spent is 23 minutes. The temperature change of the radiated material during this period of time is shown in FIG. 6. Further, the treatment amount of the radiated material including the weight of solvent is 13.4 kg/hour.

The treated substance which is discharged outside of the device is centrifuged to separate the solvent, then washed with acetone and water, then conduct quantitative analysis of the amount of holocellulose in the treated substance. Then the saccharification treatment of the treated substance is conducted. The treated substance is picked 0.5 g of dried weight to conduct quantitative analysis of holocellulose by a sodium chlorite method. Then saccharification treatment of the treated substance is conducted. In a centrifuge tube, gather dry weight 0.2 g of treated substance. Add 8 FPU of “Meicelase” (registered trademark, Meiji Seika Company) that is cellulase, and 1M of acetic acid buffer solution (pH 4.5), make a final concentration of 50 mM and a total amount of 10 mL (solid portion 2%). Infiltrate at a condition of 48 hours at 45° C. At the end of the reaction the amount of saccharides produced in the reaction liquid is measured by the Somogyi-Nelson method, calculate the rate of saccharification. As the result the rate of saccharification corresponding to holocellulose is 88.8%.

### Comparison 1

1 L of batch type microwave radiating device is used in the microwave treatment of the comparison. The treatment of radiated material is performed by the same method as the example 1, and radiated material including solvent in a total weight of 1080 g is treated. To match the highest temperature of microwave irradiation of the example 1, the radiated material is heated to 190° C. within 12 minutes, and kept at 190° C. for 15 minutes. The temperature change of the radiated material of the comparison 1 is shown in FIG. 6. The frequency of the radiating microwave is 2450 MHz; output is 1.5 kW; the pressure inside the reaction container is 0.2 MPa.

Concerning the treated substance which is obtained from the microwave treatment of the comparison 1, quantitative analysis of the amount of holocellulose and saccharification treatment are conducted using the same methods as the example 1. The rate of saccharification corresponding to holocellulose is 86.8%, less than the rate of saccharification of the example 1.

As described above microwave treatment at high temperature, high pressure may be performed and high a saccharification rate of the treated substance may be obtained by using the microwave radiating device and the connecting type microwave radiating device according to the present invention. Further, as shown in FIG. 6 compared to the microwave radiating device used in comparison 1, in the microwave radiating device of the present invention the radiated material may be heated in shorter time.

In the following examples, the method of producing a sugar ingredient from a plant material of the present invention will be described in more detail, but specification and examples are to be regarded in an illustrative rather than a restrictive sense, and all modifications are intended to be included within the scope of the present invention.

### Example 1-7 and Comparison 1

The effect of various Lewis acid catalysts which are used in the method of producing a sugar ingredient from a plant material will be discussed.

### Preparation of the Plant Material

The lumber of a beech tree which is the raw material of the plant material is crushed in a wood shredder and a Wiley mill, divided with a sieve; powder (beech tree powder) of a size 355 μm to 500 μm is collected. The beech tree powder is mixed with a mixture of ethanol/benzene (1/2 by volume) which is 2 times by volume of the volume of the beech tree powder, then refluxed. The hydrophobic low molecule substances are removed by refluxing. Next the beech tree powder is mixed with water which is 2 times by volume of the volume of the beech tree powder, and treated at 121° C., about 30 minutes in an autoclave. The hydrophilic low molecule substances are removed. Further, put the beech tree powder in a drying machine at 105° C. overnight. The prepared beech tree powder is used as plant material, and is used in examples and comparisons in the following.

### Separation Process

Put 1 g of the prepared beech tree powder, and 20 g of 1-propanol/water (1/1 by volume) to be the dispersing medium into a vial for microwave radiating; in Example 1-7, add separately 60 μmol of various Lewis acid catalysts as listed in Table 1; obtain dispersing liquids. The comparison 1 has no catalyst added. The above vials are tightly sealed; while stirring with a stirrer at 900 rpm, use a microwave radiating device called “Initiator 60” (manufactured by Biotage Japan Company) which irradiates with microwaves of 2450 MHz at a condition of 30 minutes, 180° C. to the above each dispersing liquids.

### Extraction Process

After microwave radiation the liquid is filtered by using filter paper “Advantec No. 131” (registered trademark, manufactured by Toyo Filter Paper Company, holding particle diameter 3 μm); an insoluble component which contains solid state polysaccharides and remaining lignin is obtained. The wet weight of the insoluble component is measured, then the water is removed completely by drying the component, determining the hydration rate. The weight of the insoluble component is calculated by deducting the weight of water from the wet weight; the ratio (the yield of the insoluble component) of the weight of the insoluble component and the weight of the used wood powder is calculated.

### Enzymatic Saccharification Treament

In a centrifuge tube, gather dry weight 0.2 g of treated substance; add 8 FPU of “Meicelase” (registered trademark, Meiji Seika Company) that is cellulase and 10 ml of 50 mM of acetic acid buffer solution (pH 4.5); infiltrate 48 hours at 45° C., 140 rpm. At the end of the reaction the amount of saccharides produced in the reaction is measured by the Somogyi-Nelson method. Calculate the saccharide yield corresponding to the unit weight of insoluble component and the saccharide yield corresponding to the unit weight of the plant material.

As indicated in Table 1, compared with the comparison 1 the examples 1-7 have a lower yield of insoluble component, but have a higher saccharide yield corresponding to the unit weight of insoluble component and a higher saccharide yield corresponding to the unit weight of the plant material.

TABLE 1

| | | | Saccharide yield | Saccharide yield |
|---|---|---|---|---|
| | | Yield of | corresponding to | corresponding to |
| | | insoluble | the unit weight | the unit weight |
| | | compo- | of insoluble | of the plant |
| | | nent (%) | component (%) | material (%) |
| Compar- | No | 89.5 | 16.7 | 15.0 |
| ison 1 | catalyst | | | |
| Example 1 | Aluminum | 47.8 | 88.9 | 42.5 |
| | chloride | | | |
| | [AlCl₃] | | | |
| Example 2 | Aluminum | 45.9 | 94.1 | 43.2 |
| | bromide | | | |
| | [AlBr₃] | | | |
| Example 3 | Aluminum | 51.0 | 90.6 | 46.2 |
| | sulfate | | | |
| | [Al₂ | | | |
| | (SO₄)₃] | | | |
| Example 4 | Ferric(III) | 52.1 | 88.1 | 45.9 |
| | chloride | | | |
| | [FeCl₂] | | | |
| Example 5 | Zinc | 82.6 | 41.8 | 34.5 |
| | chloride | | | |
| | [ZnCl₂] | | | |
| Example 6 | Zinc | 70.3 | 57.4 | 40.4 |
| | bromide | | | |
| | [ZnBr₂] | | | |
| Example 7 | Zinc | 80.2 | 41.9 | 33.6 |
| | sulfate | | | |
| | [Zn SO₄] | | | |

### Example 8 and Comparison 2

In a method of producing a sugar ingredient from a plant material, with a comparison of microwave treatment and external heating treatment will now be performed. In a microwave radiating vial put 1 g of the prepared beech tree powder and 20 g of 1-propanol/water (1/1 by volume) as a dispersing medium; add 60 μmol of aluminum sulfate as Lewis acid; obtain a dispersing liquid. The above vial is tightly sealed; and while stirring with a stirrer at 900 rpm, use a microwave radiating device “Initiator 60” (Biotage Japan) which irradiates microwaves of 2450 MHz at two conditions of 30 minutes, 160° C. and 30 minutes, 180° C. to irradiate the above dispersing liquid (example 8).

Further, regarding the comparison of the above dispersing liquid, which is put in an autoclave cylinder. External heating is conducted in the autoclave at two conditions of 30 minutes, 160° C. and 30 minutes, 180° C. (comparison 2). The enzymatic saccharification treatment is performed by using 1 FPU and 8 FPU of “Meicelase” (registered trademark) to conduct enzymatic saccharification of the example 8 and the comparison 2 respectively.

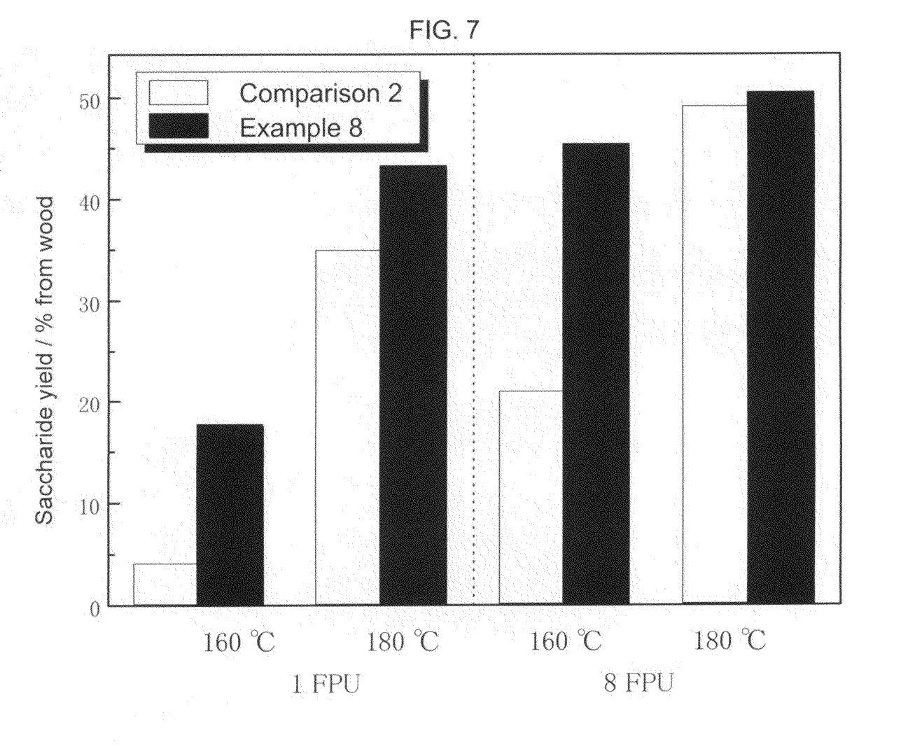

FIG. 7 indicates the saccharide yield corresponding to the weight of plant material of the example 8 and the comparison 2. No matter at what condition, the yield of the example 8 being treated by microwaves is higher. Especially the difference is obvious at the condition of a small amount of enzyme. Therefore, according to the present invention it is able to obtain saccharides with a high yield even though the amount of enzyme used for saccharification of the treated substance is small; thus drastically cutting down the saccharification cost.

### Example 9 and Comparison 3

The gas chromatography mass spectrometry of a soluble component was performed. In a microwave radiating vial put 1 g of the prepared beech tree powder to be plant material, and 20 g of 1-propanol/water (1/1 by volume) to be dispersing medium; add 60 μmol of aluminum chloride as a Lewis acid catalyst; obtain a dispersing liquid. The above vials are tightly sealed, stirring with a stirrer at 900rpm, use a microwave radiating device “Initiator 60” (made by Biotage Japan Company) which irradiates microwaves of 2450 MHz at a condition of 30 minutes, 180° C. to the above dispersing liquid (example 9). Moreover, the above dispersing liquid is put in an autoclave cylinder. External heating is conducted in an autoclave at a condition of 30 minutes, 180° C. (comparison 3).

After microwave treatment and an external heating treatment, the reaction liquid which is filtered by using a filter paper “Advantec No. 131” (registered trademark, manufactured by Toyo Filter Paper Company, holding particle diameter 3 μm), is conducted a gas chromatography mass spectrometry (GCMS).

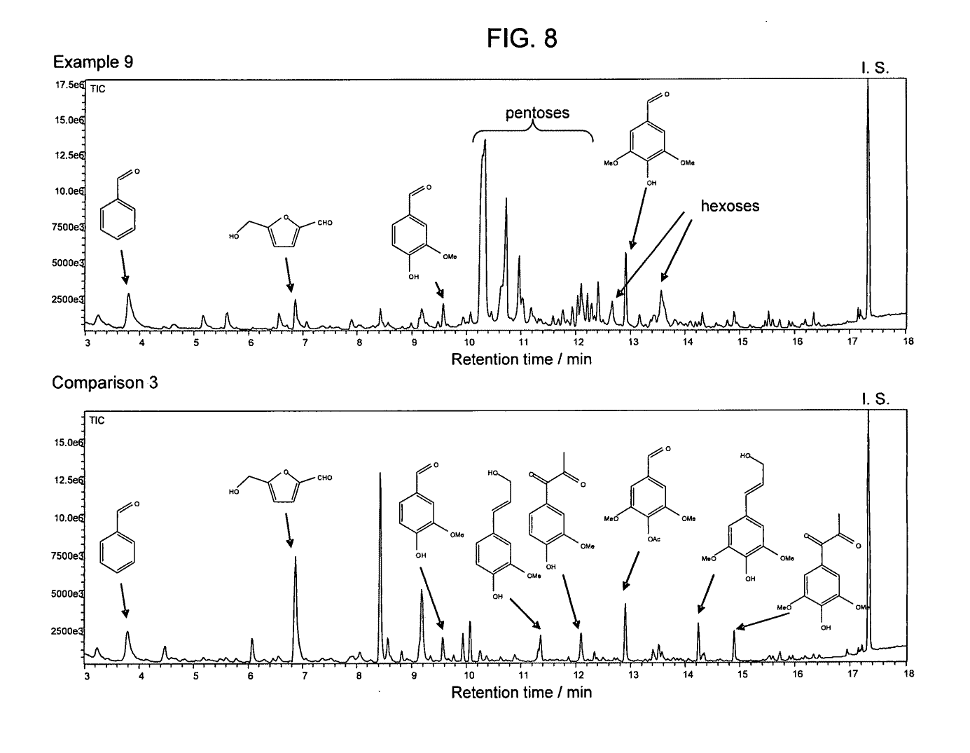

The FIG. 8 indicates the result of GCMS. Both of the example 9 and the comparison 3 contain aromatic compounds of a lignin source. The example 9, in addition to aromatic compounds, also contains much saccharide such as pentose or hexose, while the comparison 3 contains much decomposed product of saccharide such as 5-hydroxymethylfurfural (Retention time: 6.8 min-7.0 min). Consequently, it is understood that saccharide is over-decomposed due to external heating in the comparison 3. However, according to the present invention those saccharides may be utilized.

Formation of 5-hydroxymethylfurfural is inhibited by microwave treatment. 5-hydroxymethylfurfural may obstruct ethanol fermentation of saccharides. Therefore, the present invention is able to inhibit the formation of 5-hydroxymethylfurfural, and to conduct enzymatic saccharification or ethanol fermentation efficiently.

### Example 10-17 and Comparison 4

Regarding lignin of conifer (cryptomeria) which is hard to decay, the effect of the present invention is verified.

### Preparation of Plant Material

The lumber of cryptomeria which is a raw material of the plant material is crushed in a wood shredder and a Wiley mill, and divided with a sieve; powder (cryptomeria powder) of size 500 μm to 1190 μm is collected. The cryptomeria powder prepared above is used as plant material in the following examples and comparisons. The plant material used in these examples is different from the examples 1-9; the hydrophobic low molecule and hydrophilic low molecule are not removed, production of saccharide is more difficult.

### Separation Process

Put 1 g of the prepared cryptomeria powder, and 20 g of 1-propanol/water (1/1 by volume) to be dispersing medium into a vial for microwave radiating; in Example 10-17, add ammonium chloride to be the Lewis acid catalyst separately in various amounts corresponding to 1 g of plant material as listed in Table 2; obtain dispersing liquids. The comparison 4 has no catalyst added. The above vials are tightly sealed; while stirring with a stirrer at 900 rpm, use a microwave radiating device “Initiator 60” (manufactured by Biotage Japan Company) which irradiates microwaves of 2450 MHz at a condition listed in Table 2.

Extraction Process After microwave radiation the liquid is filtered by using filter paper “Advantec No. 131” (registered trademark, manufactured by Toyo Filter Paper Company, holding particle diameter 3 μm); an insoluble component is obtained. The wet weight of the insoluble component is measured, then the water is removed completely by drying the component, find out the hydration rate. The weight of the insoluble component is calculated by deducting the weight of water from the wet weight; the ratio (the yield of the insoluble component) of the weight of the insoluble component and the weight of used plant material is calculated.

### Enzymatic Saccharification Treament

In a centrifuge tube, gather dry weight 0.2 g of treated substance; add 8 FPU of “Meicelase” (registered trademark, Meiji Seika Company) that is cellulase and 10 ml of 50 mM of acetic acid buffer solution (pH 4.5); infiltrate 48 hours at 45° C., 140 rpm. At the end of the reaction the amount of saccharides produced in the reaction is measured by the Somogyi-Nelson method. Calculate the saccharide yield corresponding to the unit weight of insoluble component and the saccharide yield corresponding to the unit weight of the plant material.

The result is shown in Table 2. The yield of insoluble component of the comparison 4 is high, but the saccharide produced by enzymatic saccharification treatment is merely a small amount. The lignin contained in cryptomeria powder is considered to be hardly decomposed. On the other hand, regarding the examples 10-17, through enzymatic saccharification treatment, the saccharide yield corresponding to the unit weight of insoluble component is 75%-100% and the saccharide yield corresponding to the unit weight of the plant material is 43%-60%. Especially at the condition of 120 μm-360 μm of catalyst, saccharide may be obtained in high yield. Therefore, the present invention indicates that it is effective to produce saccharide from conifer while the traditional method indicates difficulty.

TABLE 2

| | | | | | Saccharide | |
|---|---|---|---|---|---|---|
| | | | | | yield | Saccharide |
| | | | | | corresponding | yield |
| | | | | | to the | corresponding |
| | | | | Yield of | unit weight | to the |
| | | Reaction | Reaction | insoluble | of insoluble | unit weight |
| | Catalyst | temperature | time | component | component | of the plant |
| | (μmol) | (° C.) | (min) | (%) | (%) | material (%) |
| Comparison 4 | No | 80 | 30 | 99.4 | 8.6 | 0.6 |
| | catalyst | | | | | |
| Example | 60 | 180 | 30 | 52.6 | 82.1 | 43.2 |
| 10 | | | | | | |
| Example | 120 | 180 | 20 | 45.1 | 96.2 | 43.4 |
| 11 | | | | | | |
| Example | 180 | 180 | 10 | 46.2 | 98.3 | 45.4 |
| 12 | | | | | | |
| Example | 180 | 160 | 30 | 49.5 | 99.8 | 49.4 |
| 13 | | | | | | |
| Example | 180 | 160 | 20 | 53.0 | 88.5 | 46.9 |
| 14 | | | | | | |
| Example | 360 | 150 | 30 | 56.4 | 100.0 | 56.4 |
| 15 | | | | | | |
| Example | 360 | 150 | 20 | 59.8 | 79.3 | 47.4 |
| 16 | | | | | | |
| Example | 720 | 140 | 30 | 63.9 | 75.3 | 48.1 |
| 17 | | | | | | |

Moreover, on the basis of the examples, the present invention will be described in further detail, but the specification and examples are to be regarded in an illustrative rather than a restrictive sense, and all modifications are intended to be included within the scope of the present invention.

<<Selection of Ion Used Together with Ammonium Ion>>

In the method of producing a sugar ingredient from a plant material, regarding the kind of ions to be used together with ammonium ion and peroxide will be studied below. The plant material is treated by heating externally in the presence of various ammonium salts and hydrogen peroxide, and the saccharide yield is calculated.

### Preparation of Plant Material

The lumber of beech tree which is a raw material of the plant material is crushed in a wood shredder and a Wiley mill, divided with a sieve; powder (beech tree powder) of size 355 μm to 500 μm is collected. The prepared beech tree powder is the plant material and is used in the following examples and comparisons.

### Separation Process

Put 1 g the prepared beech tree powder and 20 g water as a dispersing medium into a flask; add ammonium salt listed in Table 1 separately, to make each final concentration of 1 mM (therein, “no catalyst” is “no ammonium salt added”); then add hydrogen peroxide to make a final concentration of 0.88M; obtain dispersing liquids (A)-(J). The above flasks are tightly sealed; and while stirring with a stirrer, are conducted external heating treatments by a water bath at 80° C., 5hours.

### Extraction Process

After a separation process the liquid is filtered by using a filter paper “Advantec No. 131” (registered trademark, manufactured by Toyo Filter Paper Company, holding particle diameter 3 μm); an insoluble component which contains solid state polysaccharide and remaining lignin is obtained. The wet weight of the insoluble component is measured, then the water is removed completely by drying the component, determining the hydration rate. The weight of the insoluble component is calculated by deducting the weight of water from the wet weight; the ratio (the yield of the insoluble component) of the weight of the insoluble component and the weight of the used wood powder is calculated.

### Enzymatic Saccharification Treament

In a centrifuge tube, gather dry weight 0.2 g of treated substance; add 8 FPU of “Meicelase” (registered trademark, Meiji Seika Company) that is cellulase and 10 ml of 50 mM of acetic acid buffer solution (pH 4.5); infiltrate 48 hours at 45° C., 140 rpm. At the end of the reaction the amount of saccharides produced in the reaction liquid is measured by the Somogyi-Nelson method. Calculate the saccharide yield corresponding to the unit weight of the insoluble component and the saccharide yield corresponding to the unit weight of the plant material.

As shown in the following Table 3, (A) ammonium molybdate has the highest saccharide yield compared with other ammonium salts. Apropos of (B) ammonium phosphate dibasic or (C) ammonium hydrogen carbonate, (D) ammonium carbonate also has a higher saccharide yield. From these results it is considered that through the separation process in which molybdic acid ion, ammonium ion and peroxides are used, solid state polysaccharide which can produce saccharide efficiently is obtained.

TABLE 3

| | | | Saccharide yield | Saccharide yield |
|---|---|---|---|---|
| | | Yield of | corresponding to | corresponding to |
| | | insoluble | the unit weight | the unit weight |
| | | compo- | of insoluble | of the plant |
| | | nent (%) | component (%) | material (%) |
| (A) | Ammonium | 61.9 | 74.5 | 46.2 |
| | molybdate | | | |
| (B) | ammonium | 85.4 | 21.6 | 18.5 |
| | phosphate | | | |
| | dibasic | | | |
| (C) | Aluminum | 91.4 | 16.5 | 15.1 |
| | hydrogen | | | |
| | carbonate | | | |
| (D) | Aluminum | 76.8 | 19.1 | 14.7 |
| | carbonate | | | |
| (E) | Ammonium | 84.7 | 7.5 | 6.4 |
| | acetate | | | |
| (F) | Ammonium | 89.9 | 6.7 | 6.0 |
| | chloride | | | |
| (G) | Ammonium | 97.4 | 5.6 | 5.5 |
| | sulfate | | | |
| (H) | Ammonium | 89.4 | 6.2 | 5.5 |
| | tartrate | | | |
| (I) | Ammonium | 91.2 | 4.5 | 4.1 |
| | borate | | | |
| (J) | No catalyst | 90.0 | 5.4 | 4.9 |

### Example 1 and Comparison 1

Comparison of ammonium molybdate and other molybdates which are used in the methods of producing a sugar ingredient from a plant material according to the present invention is conducted. In a microwave radiating vial put 1 g of the prepared beech tree powder and 20 g of water as a dispersing medium; add various molybdate listed in Table 2 to make final concentration of 1 mM (therein, “no catalyst” is “no molybdate added”), and add hydrogen peroxide to make a final concentration of 0.88M; obtain dispersing liquids. The above vials are tightly sealed; and while stirring with a stirrer at 900 rpm, use a microwave radiating device “Initiator 60” (Biotage Japan) which irradiates microwaves of 2450 MHz at a condition of 30 minutes, 140° C.

Next, the above extraction process is conducted in the example 1 and the comparison 1 respectively; and the above enzymatic saccharification treatment wherein 8 FPU of “Meicelase” (registered trademark) is performed.

As shown in Table 4 below, compared with the comparisons which use other molybdate, examples which use ammonium molybdate have a higher saccharide yield corresponding to a unit weight of insoluble component and higher saccharide yield corresponding to a unit weight of plant material. Therefore, through using molybdic acid ion, ammonium ion and peroxides as disclosed in the present invention, it is able to produce a sugar ingredient which contains solid state polysaccharide and can be converted to saccharide efficiently by enzymatic saccharification treatment.

TABLE 4

| | | | Saccharide yield | Saccharide yield |
|---|---|---|---|---|
| | | Yield of | corresponding to | corresponding to |
| | | insoluble | the unit weight | the unit weight |
| | | compo- | of insoluble | of plant |
| | molybdate | nent (%) | component (%) | material (%) |
| Exam- | Ammonium | 68.7 | 90.3 | 61.9 |
| ple 1 | molybdate | | | |
| Com- | No catalyst | 61.7 | 32.2 | 19.8 |
| pari- | Sodium | 66.9 | 56.4 | 37.7 |
| son 1 | molybdate | | | |
| | Calcium | 72.5 | 39.1 | 28.3 |
| | molybdate | | | |
| | Potassium | 76.6 | 37.3 | 28.6 |
| | molybdate | | | |

### <<Relevance Between the Amount of Enzyme and the Rate of Enzymatic Saccharification>>

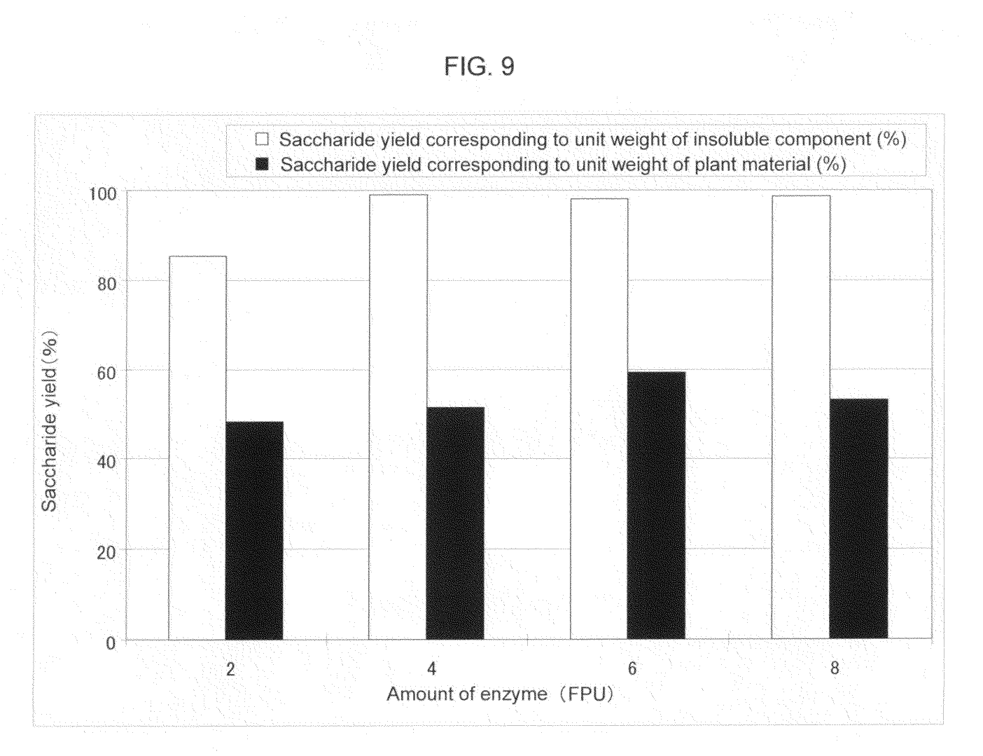

Enzymatic saccharification of using 2 FPU, 4 FPU, 6 FPU, and 8 FPU of “Meicelase” (registered trademark) is performed on the insoluble component which is obtained from the separation process wherein the same treatment as the example 1 is conducted except microwave radiating is at 160° C., 9 minutes. The saccharide yield corresponding to unit weight of insoluble component and the saccharide yield corresponding to unit weight of plant material are shown in FIG. 9. As shown in FIG. 9, high saccharide yield is obtained even at 2 FPU-4 FPU of enzyme. Therefore the solid state polysaccharide contained in insoluble component is obtained by the present invention, and is able to be saccharized even at a small amount of enzyme, thus it may achieve huge cost savings of saccharification.

### Example 2 and Comparison 2

The saccharide yield of microwave radiating (example 2) according to the present invention and external heating (comparison 2) are compared. Separation process of example 2 is performed as follows. In a microwave radiating vial put 1 g of the prepared beech tree powder and 20 g of water to be the dispersing medium; add separately ammonium molybdate to make a final concentration as entered in Table 3, and add hydrogen peroxide to make a final concentration of 0.88M; obtain dispersing liquid. The above vials are tightly sealed; and while stirring with a stirrer at 900 rpm, use a microwave radiating device “Initiator 60” (Biotage Japan) which irradiates microwaves of 2450 MHz at a condition of 30 minutes, 140° C. to the above dispersing liquid.

Further, the separation process of the comparison 2 is performed as follows. In an autoclave cylinder put 1 g of the prepared beech tree powder and 20 g of water to be the dispersing medium; add separately ammonium molybdate to make a final concentration as listed in Table 3, and add hydrogen peroxide to make a final concentration of 0.88M; obtain dispersing liquids. The above cylinders are heated externally by using an autoclave at condition of 30 minutes, 140° C.

Next, the above extraction process is conducted on mixtures obtained in example 2 and the comparison 2, respectively; and the above enzymatic saccharification treatment wherein 8 FPU of “Meicelase” (registered trademark) is used, is performed.

As shown in Table 5 below, no matter at any concentration of ammonium molybdate, the examples, which are treated with microwave radiating, have a higher saccharide yield compared with the comparisons which are heated externally.

TABLE 5

| | | | | Saccharide yield | Saccharide yield |
|---|---|---|---|---|---|
| | | Final conc. | Yield of | corresponding to | corresponding to |
| | | of ammonium | insoluble | the unit weight | the unit weight |
| | Method of | molybdate | component | of insoluble | of plant |
| | heating | (mM) | (%) | component (%) | material (%) |
| Example 2 | Microwave | 0.25 | 63.5 | 21.1 | 13.3 |
| | radiating | 0.5 | 59.8 | 72.4 | 41.8 |
| | | 0.75 | 55.7 | 85.9 | 47.9 |
| | | 1.0 | 44.9 | 85.0 | 38.7 |
| Comparison 2 | Externally | 0.25 | 60.2 | 16.8 | 10.8 |
| | heating | 0.5 | 45.8 | 64.7 | 29.5 |
| | | 0.75 | 43.4 | 78.1 | 33.9 |
| | | 1.0 | 47.8 | 43.8 | 20.9 |

### Example 3 and Comparison 3

Regarding to lignin of conifer (cryptomeria) which is hard to decay, the effect of the present invention is verified.

### Preparation of Plant Material

The lumber of cryptomeria which is a raw material of the plant material is crushed in a wood shredder and a Wiley mill, and divided with a sieve; powder (cryptomeria powder) of a size 500 μm to 1190 μm is collected. The cryptomeria powder prepared like above is used as a plant material in the following example and comparison.

Separation process of the example 3 is performed through irradiating microwaves at the same condition as the example 2, except using 1 g of the prepared cryptomeria powder, adding ammonium molybdate which has various final concentrations as listed in Table 4. Further, a separation process of the comparison 3 is performed through externally heating at the same condition as the comparison 2, except using 1 g of the prepared cryptomeria powder, adding ammonium molybdate which has various final concentrations as listed in Table 4.

Next, the above extraction process is conducted on mixtures obtained in example 3 and the comparison 3, respectively; and the above enzymatic saccharification treatment wherein 8 FPU of “Meicelase” (registered trademark) is used, is performed.

As shown in Table 6 below, no matter what concentration of ammonium molybdate is used, the examples, which are treated with microwave radiating, have a higher saccharide yield compared with the comparisons which are heated externally. Especially at existence of 2.5 mM-5.0 mM of ammonium molybdate it is still able to obtain saccharide at high yield. Therefore, the present invention indicates that it is effective to produce saccharide from conifer while the traditional method indicates difficulty.

TABLE 6

| | | | | Saccharide yield | Saccharide yield |
|---|---|---|---|---|---|
| | | Final conc. | Yield of | corresponding to | corresponding to |
| | | of ammonium | insoluble | the unit weight | the unit weight |
| | Method of | molybdate | component | of insoluble | of plant |
| | heating | (mM) | (%) | component (%) | material (%) |
| Example 3 | Microwave | 1.0 | 63.5 | 21.1 | 13.3 |
| | radiating | 2.5 | 59.8 | 72.4 | 41.8 |
| | | 3.75 | 55.7 | 85.9 | 47.9 |
| | | 5.0 | 44.9 | 85.0 | 38.7 |
| Comparison 3 | Externally | 1.0 | 60.2 | 16.8 | 10.8 |
| | heating | 2.5 | 45.8 | 64.7 | 29.5 |
| | | 3.75 | 43.4 | 78.1 | 33.9 |
| | | 5.0 | 47.8 | 48.8 | 20.9 |

In the following the most appropriate conditions concerning the present invention will be discussed.

### <<Consideration Concerning the Most Appropriate Concentration of Hydrogen Peroxide>>

In the above separation process, the dispersing liquids of (A)-(D) are obtained through adding ammonium molybdate to make a final concentration of 1 mM, and adding hydrogen peroxide individually of a concentration as listed in Table 7. The above vials are tightly sealed; and while stirring with a stirrer at 900 rpm, use a microwave radiating device “Initiator 60” (Biotage Japan) which irradiates microwaves of 2450 MHz at condition of 30 minutes, 140° C. to the above dispersing liquids. The obtained insoluble component is extracted byan extraction process, and is conducted to an enzymatic saccharification treatment through using 8 FPU of “Meicelase” (registered trademark).

The results are shown in the following Table 7. At the condition that the final concentration of (D) hydrogen peroxide is 0.88M, the highest saccharide yield is obtained.

TABLE 7

| | | | Saccharide yield | Saccharide yield |
|---|---|---|---|---|
| | Final | Yield of | corresponding to | corresponding to |
| | concentration | insoluble | the unit weight | the unit weight |
| | of hydrogen | component | of insoluble | of the plant |
| | peroxide (M) | (%) | component (%) | material (%) |
| (A) | 0.22 | 81.7 | 47.9 | 39.1 |
| (B) | 0.44 | 76.9 | 56.9 | 43.8 |
| (C) | 0.66 | 64.5 | 77.6 | 49.4 |
| (D) | 0.88 | 67.4 | 80.0 | 59.8 |

### <<Consideration Concerning the Most Appropriate Reaction Time>>

In the above separation process, the dispersing liquids are obtained through adding ammonium molybdate to make a final concentration of 1 mM, and adding hydrogen peroxide individually of a concentration of 0.88M. The above vials are tightly sealed; and while stirring with a stirrer at 900 rpm, use a microwave radiating device “Initiator 60” (Biotage Japan) which irradiates microwaves of 2450 MHz to the above dispersing liquids at a reaction temperature of 140° C., reaction time as listed in Table 8. The obtained insoluble component is extracted by an extraction process, and is conducted to enzymatic saccharification treatment through using 8 FPU of “Meicelase” (registered trademark).

The results are shown in the following Table 8. At the condition that (C) reaction temperature is 30 minutes, the highest saccharide yield is obtained.

TABLE 8

| | | | Saccharide yield | Saccharide yield |
|---|---|---|---|---|
| | | Yield of | corresponding to | corresponding to |
| | Reaction | insoluble | the unit weight | the unit weight |
| | time | component | of insoluble | of the plant |
| | (minute) | (%) | component (%) | material (%) |
| (A) | 10 | 75.7 | 25.6 | 19.5 |
| (B) | 20 | 68.7 | 44.9 | 30.9 |
| (C) | 30 | 67.4 | 88.7 | 59.8 |
| (D) | 40 | 50.6 | 88.5 | 44.0 |
| (E) | 60 | 49.8 | 88.0 | 43.9 |

In accordance with the present invention the method of separating a sugar ingredient from a plant material may make a contribution to the realization of a biofinery that obtains useful chemical substances such as ethanol or aromatic compounds etc. from biomass raw material. Moreover, the present invention may also apply to reducing plant material to pulp or bleaching of pulp.

1-16. (canceled)

17. A microwave radiating device which treats a material by radiating microwaves to the material, comprising:
a radiating means containing a microwave radiating source;
a container which accommodates the material therein; the container having:
a supply means for providing the material to the container,
a discharge means for discharging the material from the container, and
a microwave receiving section where the microwave enters the container through a dielectric material; and
at least one layer composed of a non-dielectric material, the layer provided between an inside of the container and an atmosphere outside of the container;
wherein the radiating means and the container are detachably connected.

18. The microwave radiating device of claim 17, wherein a sheet-shaped member is laminated on the dielectric material in a microwave-radiating direction.

19. The microwave radiating device of claim 17, wherein the dielectric material is sandwiched with at least two sheet-shaped members.

20. The microwave radiating device of claim 17, wherein the dielectric material is made of at least one substance selected from the group consisting of: quartz glass, fluorocarbon polymers, ceramics, alumina, sapphire, and diamond; or
the non-dielectric material is made of at least one substance selected from the group consisting of: stainless steel, aluminium, titanium, nickel, gold, and silver.

21. A connecting-type microwave radiating device, comprising:
at least two microwave radiating devices, the microwave radiating devices coupled to each other, and each of the microwave radiating devices comprising;
a radiating means containing a microwave radiating source;
a container which accommodates a material therein; the container having:
a supply means for providing the material to the container,
a discharge means for discharging the material from the container, and
a microwave receiving section where the microwave enters the container through a dielectric material;
at least one layer composed of a non-dielectric material, the layer provided between an inside of the container and an atmosphere outside of the container;
wherein the radiating means and the container are detachably connected;
wherein a discharge means in one of the microwave radiating devices and a supply means in another of the microwave radiating devices adjacent to the one of the microwave radiating devices are detachable from each other, and
wherein when the discharge means and the supply means are coupled, the containers of the one and the another of the microwave radiating devices jointly form a passage for a flow of the material.

22. The connecting-type microwave radiating device of claim 21, wherein a sheet-shaped member is laminated on the dielectric material in a microwave-radiating direction.

23. The connecting-type microwave radiating device of claim 21, wherein the dielectric material is sandwiched with at least two sheet-shaped members.

24. A method for producing a sugar component from a plant material, comprising a step of:
obtaining a mixture containing a decomposed product of lignin and the sugar component containing at least a solid-state polysaccharide by irradiating the plant material containing at least lignocellulose with a microwave in a presence of a Lewis acid catalyst.

25. The method of claim 24, wherein a frequency of the microwave is in a range of 2000 MHz to 6000 MHz.

26. The method of claim 24, wherein the plant material is irradiated with the microwave for a duration ranging from 1 minute to 60 minutes.

27. The method of claim 24, wherein the plant material is irradiated with the microwave so that a temperature of the plant material ranges from 80° C. to 240° C.

28. The method of claim 24, wherein the Lewis acid catalyst is made of at least one substance selected from the group consisting of: aluminium chloride, aluminium bromide, aluminium sulfate, iron(III) chloride, zinc chloride, zinc bromide, and zinc sulfate.

29. A method for producing a sugar component from a plant material, comprising a step of
obtaining a mixture containing a decomposed product of lignin and the sugar component containing at least a solid-state polysaccharide by irradiating the plant material containing at least lignocellulose with a microwave in a presence of a molybdic acid ion, an ammonium ion, and a peroxide.

30. The method of claim 29, wherein the peroxide is a hydrogen peroxide.

31. The method of claim 29, wherein a frequency of the microwave ranges from 2000 MHz to 6000 MHz.

32. The method of claim 29, wherein the plant material is irradiated with the microwave for a duration ranging from 1 minute to 60 minutes.

33. The method of claim 29, wherein the plant material is irradiated with the microwave so that a temperature of the plant material ranges from 50° C. to 240° C.

34. The method of claim 29, wherein the plant material is irradiated with the microwave in a condition that the plant material is dispersed in a water.
